(19) United States
(12) Patent Application Publication
(10) Pub. No.: US 2013/0316020 A1
(43) Pub. Date: Nov. 28, 2013

(54) WOUND DRESSINGS COMPRISING CHLORITE

(71) Applicants: Servet Buyuktimkin, San Diego, CA (US); Nadir Buyuktimkin, San Diego, CA (US); Edward T. Kisak, San Diego, CA (US); Dominic King-Smith, San Diego, CA (US); John M. Newsam, La Jolla, CA (US); Jagat Singh, Toronto (CA)

(72) Inventors: Servet Buyuktimkin, San Diego, CA (US); Nadir Buyuktimkin, San Diego, CA (US); Edward T. Kisak, San Diego, CA (US); Dominic King-Smith, San Diego, CA (US); John M. Newsam, La Jolla, CA (US); Jagat Singh, Toronto (CA)

(73) Assignee: NUVO RESEARCH AG

(21) Appl. No.: 13/984,443

(22) PCT Filed: Feb. 9, 2012
(86) PCT No.: PCT/CA2012/050072

§ 371 Date: Aug. 8, 2013

Related U.S. Application Data
(60) Provisional application No. 61/441,016, filed on Feb. 9, 2011.

(51) Int. Cl. A61L 15/44

(52) U.S. Cl. CPC A61L 15/44
USPC 424/665; 424/661

(57) ABSTRACT

This application described a wound dressing comprising an absorbent material, an effective amount of chlorite and an amount of a base to provide a pH of the dressing material greater than or equal to about (10). Advantageously, the wound dressings of the present application provide a convenient source of chlorite that is stable and easy to use.

The present application claims the benefit of priority of co-pending U.S. provisional patent application No. 61/441,016 filed on Feb. 9, 2011, the contents of which are incorporated herein by reference in their entirety.

### FIELD OF THE APPLICATION

The present application relates to wound dressings comprising chlorite, methods for their preparation and their topical use, for example, for the treatment of wounds.

### BACKGROUND OF THE APPLICATION

### Wound Treatments

Chronic, hard-to-heal wounds are a serious problem with an increasing incidence. Chronic wounds can be caused by such conditions as pressure sores and poor circulation in the lower extremities. Co-morbid conditions, such as diabetes and atherosclerosis, reduce blood flow to the extremities and also increase the likelihood of developing chronic wounds. Wound infections following a breakdown of surgical or traumatic wounds also continue to pose a serious concern.

Antibiotics, both systemically or topically administered, represent a milestone in the treatment of infected wounds. However, antibiotics, per se, may represent a “toxic” burden to a patient with multiple injuries, deeps burns, or stressed liver function. As well, administration of antibiotics may result in the formation of resistant bacterial strains, preventing additional treatment with antibiotics.

The use of an aqueous solution containing a stabilized chlorite solution for treating wounds and infections is described in Hinz et al., The Lancet (1986), U.S. Pat. Nos. 4,507,285 and 4,725,437, and EP 0 200 157. These documents describe the use of a stabilized chlorite solution in stimulating the wound healing response in humans, as well as in treating infections caused by parasites, fungi, bacteria, viruses and/or mycoplasma.

Oxovasin™, also sold under the brand name Oxoferin™, is one commercially available stabilized chlorite solution known for its antimicrobial properties. In Germany, Oxovasin™ is indicated for the treatment of wounds and wound healing disorders by improvement of wound cleansing, of granulation, of epithelization and of wound closure, and has demonstrated antimicrobial activity in vitro (Teepe et al., J. of Trauma 35(1):8-19, 1993).

### Buffered Chlorite Solutions

U.S. Pat. No. 5,855,922 discloses a skin treatment solution comprising a metal chlorite in a concentration of from about 0.002% (20 ppm) to about 0.5% (5000 ppm). The solution has a pH in the range of about 6 to about 10 via the use of a buffer. It is taught that these compositions may be applied in the form of a gel, with cellulose gels (e.g. methyl, hydroxy methyl and hydroxy ethyl cellulose) being preferred, or incorporated into a variety of materials to produce wound dressings. No actual dressings were prepared and tested.

U.S. patent application publication no. 2007/0145328 discloses chlorite formulations for parenteral, systemic or intravenous administration comprising chlorite and a pH adjusting agent to adjust the pH in the range of about 7 to about 11.5).

### Topical Wound Dressings

U.S. Pat. No. 5,116,620 describes an antimicrobial wound dressing comprising two adjacent layers. One layer is impregnated with lyophilized, stabilized chlorine-containing compounds which generate, on activation, chlorine dioxide, and the adjacent layer comprises a dry, activating amount of an acidic compound. This multi-layered wound dressing is, however, difficult to manufacture, unstable unless lyophilized, and requires wound moisture for activation. Accordingly, the dressing does not inherently possess antimicrobial properties, nor upon application to a non- or light-exudating wound is it effective as an antimicrobial agent

U.S. Pat. No. 5,133,965 discloses a sustained-release wound dressing material comprised of a foam bandage material and a precursor solution for vehicles called solvent dilution microcarriers (SDMCs) which encapsulate passenger molecules. The encapsulating vehicles are formed using a multistep method that first involves preparation of a “formed solution”, followed by an organization step which results in the creation of the SDMCs from the “formed solution”. It is the “formed solution” that is absorbed onto the dressing material. The “formed solution” is prepared by dissolving an amphiphatic material and a passenger molecule in an organic solvent, followed by addition of water to obtain a turbid solution and then addition of more organic solvent to obtain the clear “formed solution”. In the SDMCs, the passenger molecule is entrapped in the bilayer itself, or in association with a component of the bilayer, rather than inside the space created by a spherical bilayer. Among the carrier molecules that were encapsulated in an SDMC was tetrachlorodecaoxide (TCDO).

Weise K. and Evers, K. H. (Actuelle Traumatologie, 1988, 18:219-225) describes the treatment of difficult wounds in patients using TCDO-impregnated dressings. No pretreatment of the dressings was reported and no statistically significant results were observed.

### SUMMARY OF THE APPLICATION

The present application relates to wound dressings comprising chlorite and methods of using these materials to treat wounds. In particular the present application relates to a topical wound dressing material comprising chlorite, for use in wound healing. The challenge has been to develop a dressing in which the chlorite will remain active and the dressing material does not degrade due to reactivity of the chlorite.

It has been found that pre-treatment of an absorbent material with a base to provide a pH that is greater than or equal to about 10 prior to impregnation of the material with the chlorite provides dressing materials that are stable and maintain the activity of the chlorite.

Accordingly, the present application includes a wound dressing comprising an absorbent material, an effective amount of chlorite and an amount of a base to provide a pH of the absorbent material greater than or equal to about 10.

In an embodiment, the chlorite is added to the absorbent material as an aqueous composition comprising chlorite. Non-limiting examples of commercially available chlorite compositions include WF10, Oxovasin™, and OXO-K993. Other non-limiting chlorite-based compositions are described in U.S. Pat. Nos. 6,350,438, 6,251,372, 6,235,269, 6,132,702, 6,077,502, 5,820,822 and 4,574,084, the contents of which are incorporated herein by reference. Also included are compositions comprising chlorite salts such as sodium chlorite.

The base can be any suitable base that is compatible for use with the absorbent materials and the chlorite.

Suitable bases include, for example, organic and inorganic bases, such as alkali metal bases, alkaline earth metal bases, amine bases (such as alkylamines, ammonia and ammonia hydroxide), amide bases and methyloxide bases.

In a further embodiment, the wound dressing further comprises a color stabilizing agent, such as, arginine.

In a further embodiment, the wound dressing is partially saturated with a chlorite solution.

In a further embodiment, the wound dressing is fully saturated with a chlorite solution.

In a further embodiment, the wound dressing is suitably thick and has an areal dimension that provides an absorptive capacity for handling wound exudate.

In a further embodiment, the wound dressing has an active release behavior for chlorite ions that is substantially linear.

In a further embodiment, the wound dressing comprises a single active agent-containing absorbent layer.

In a further embodiment, the wound dressing comprises antimicrobial properties and does not allow microbial growth or proliferation once prepared.

In a further embodiment, the present application includes a wound dressing that is stable, comprises desirable antimicrobial properties and is useful in treating a wide variety of wounds. In another embodiment of the application, the wound dressing is effective upon application (i.e. is not activated in situ) and is useful in the wound healing process.

In another embodiment, the application includes a wound dressing comprising a chlorite ion concentration that decomposes by less than about 5% over the course of at least about 6 months at about room temperature. In another embodiment, the rate of decomposition is less than about 4.5%, 4%, 3.5%, 3.0%, 2.5%, 2%, 1.5%, 1.0%, or less than about 0.5%, and all fractions in between, over the course of at least six months at room temperature.

The present application also includes a wound dressing that is isotonic with a subject's body fluids.

The present application also includes a method for preparing a wound dressing comprising:
(a) treating an absorbent material with a base solution to provide a pH that is greater than or equal to about 10; and
(b) treating the absorbent material with an effective amount of chlorite,
wherein the absorbent material is treated with the chlorite concurrently or after treatment with the base.

The present application also includes a method for preparing a wound dressing comprising:
(a) treating an absorbent material with a base solution to provide a pH that is greater than or equal to about 10;
(b) removing excess base solution and/or drying the absorbent material; and
(c) impregnating the absorbent material with an effective amount of chlorite.

In an embodiment, the method of preparing a wound dressing further comprises sealing the dressing in a sealable enclosure. In an embodiment, the wound dressing is moist or wet when sealed in the sealable enclosure and/or when used. In another embodiment, the wound dressing is dried prior to being sealed in the sealable enclosure and/or prior to use. In yet another embodiment, the wound dressing is not lyophilized prior to being sealed in the sealable enclosure and/or prior to use.

The present application further includes a wound dressing prepared by a process comprising:
(a) treating an absorbent material with a base solution to provide a pH that is greater than or equal to about 10; and,
(b) treating the absorbent material with an effective amount of chlorite,
wherein the absorbent material is treated with the chlorite concurrently or after treatment with the base.

In a further embodiment, the wound dressing is packaged in a suitable sealable enclosure. Accordingly, in a further embodiment of the application, there is included a pharmaceutical package comprising:
(a) a wound dressing comprising an absorbent material, an effective amount of chlorite and an amount of a base to provide a pH of the absorbent material greater than or equal to about 10;
(b) a sealable enclosure for the wound dressing; and
(c) optionally, instructions for use.

In an embodiment, the pharmaceutical package comprises a single active agent-containing absorbent layer, wherein the active agent is chlorite.

The present application also includes a method for treating a condition comprising applying a wound dressing of the application to a subject in need thereof. In one embodiment, the wound dressing is applied to the wound of the subject. In another embodiment, the dressing is applied to a cavity wound of the subject alone, or in combination with a bandage to retain the dressing in place and/or to prevent run-off.

The application further includes a method for treating wounds comprising applying a wound dressing of the application to a subject in need thereof. In one embodiment, the wound dressing is applied to the wound or wound bed of the subject or into a wound of the subject.

The application further includes a use of a wound dressing of the application for wound healing. Wound healing includes, for example, pressure, burn, post-operative or post-traumatic wound healing, or chronic wound healing as in the healing of diabetic ulcers, venous ulcers, arterial ulcers or decubitus ulcers. The dressing is also suitable for use on full thickness wounds (e.g. Stage III or V ulcers) with light, moderate or heavy exudates as well as non exudating wounds.

The present application also includes a use of arginine as a color stabilizing agent in a wound dressing, the wound dressing comprising an absorbent material, an effective amount of chlorite and an amount of a base to provide a pH of the absorbent material greater than or equal to about 10.

Other features and advantages of the present application will become apparent from the following detailed description. It should be understood, however, that the detailed description and the specific examples, while indicating embodiments of the application, are given by way of illustration only, since various changes and modifications within the spirit and scope of the application will become apparent to those skilled in the art from this detailed description.

### BRIEF DESCRIPTION OF THE DRAWINGS

The embodiments of the application will now be described in greater detail with reference to the attached drawings in which:

FIG. 1 shows the effect of wet vs dry treatments on the pH as a function of time of sponge 1, 6 and mesh J (see Table 1A) that have been pre-treated with 0.1 M Na₂CO₃ and dosed with Oxovasin™.

FIG. 2 shows the effect of wet vs dry treatments on the pH as a function of time of sponge 1, 6 and mesh J (see Table 1A) that have been pre-treated with 0.1 M Na₂CO₃/0.1 M arginine and dosed with Oxovasin™.

FIG. 3 shows the pH profile over 20 weeks of a Medisponge™ W30 pre-treated with either 0.1 M Na₂CO₃ or 0.2 M Na₂CO₃ and dosed with Oxovasin™ then kept at room temperature (RT) or at refrigerator temperature (RF).

FIG. 4 shows the pH profile over 20 weeks of a Medisponge™ W30 pre-treated with either 0.1 M Na₂CO₃/arginine or deionized water (DI) and dosed with Oxovasin™ then kept at room temperature (RT) or at refrigerator temperature (RF).

FIGS. 5A-B show the average amount of chlorite (as determined using HPLC) over time in Oxovasin™ dosed Medisponge™ (FIG. 5A) and Stratex Sponge (FIG. 5B), that have been pre-treated with 0.1 M Na₂CO₃ or 0.2 M Na₂CO₃ alone or in combination with arginine, and then kept at room temperature (RT) or at refrigerator temperature (RF).

FIG. 6 shows the pH profile over 16 weeks of a Medisponge™ 50P pre-treated with either 0.1 M Na₂CO₃ or 0.2 M Na₂CO₃ and dosed with Oxovasin™ then kept at room temperature (RT) or at refrigerator temperature (RF).

FIG. 7 shows the pH profile over a period of up to 16 weeks of a Medisponge™ 50P pre-treated with either 0.1 M Na₂CO₃/arginine or 0.1 M Na₂CO₃ and dosed with Oxovasin™ then kept at room temperature (RT) or at refrigerator temperature (RF).

FIG. 8 shows the pH profile over 8 weeks of a Medisponge™ 50P pre-treated deionized water (DI) and dosed with Oxovasin™ then kept at room temperature (RT) or at refrigerator temperature (RF).

FIG. 9 shows the average amount of chlorite remaining in various sponges (determined using HPLC) over time after pre-treatment with 0.1 M Na₂CO₃ and dosed with Oxovasin™.

FIG. 10 is a schematic showing the color measurement system utilized in Example 4.

FIGS. 11A-B show the release profile of chlorite ions from Mirasorb gauze G, Medisponge™ 50P and Medisponge™ 30W pretreated with 0.1 M Na₂CO₃ solution and dosed with Oxovasin™. Also tested was Mirasorb gauze G that received no pretreatment with 0.1 M Na₂CO₃. The unpretreated and pretreated Mirasorb gauzes are identified as G1 and GW2, respectively.

FIG. 12 shows the average amount of chlorite in Medisponge™ 30W 0.375 inches pre-treated with 0.1 M Na₂CO₃ and dosed with 4× diluted OXO-K993 after freeze-drying for 24 hours compared to control sponges kept at room temperature for 24 hours and chlorite in an open container and freeze dried.

FIG. 13 shows the average change in pH over time of Medisponge™ 30W pretreated with ˜pH 8.5 0.01M PBS (weak base) and dosed with Oxovasin™.

FIG. 14 shows the average change in pH over time of Medisponge™ 30W pretreated with ˜pH 9.0 0.01M PBS (weak base) and dosed with Oxovasin™.

FIG. 15 shows the average change in pH over time of Medisponge™ 30W pretreated with ˜pH 9.5 0.01M PBS (weak base) and dosed with Oxovasin™.

FIG. 16 shows the average change in pH over time of Medisponge™ 30W pretreated with 0.1M PBS buffer (at pH 8.51, 9.02 and 9.54) and dosed with Oxovasin™.

FIG. 17 shows the percent drop in pH over time of Medisponge™ 30W pretreated with 0.1M PBS buffer (at pH 8.51, 9.02 and 9.54) and water and dosed with Oxovasin™.

FIG. 18 shows the average loss in weight over time of Medisponge™ 30W pretreated with 0.1M PBS buffer and dosed with Oxovasin™.

### DETAILED DESCRIPTION OF THE APPLICATION

### I. Definitions

Unless otherwise indicated, the definitions and embodiments described in this and other sections are intended to be applicable to all embodiments and aspects of the application herein described for which they are suitable as would be understood by a person skilled in the art.

The terms “a,” “an,” or “the” as used herein not only include aspects with one member, but also includes aspects with more than one member. For example, an embodiment including “a base” should be understood to present certain aspects with one base or two or more additional bases.

In compositions comprising an “additional” or “second” component, the second component as used herein is chemically different from the other components or first component. A “third” component is different from the other, first, and second components, and further enumerated or “additional” components are similarly different.

The terms “acceptable time period” or “acceptable period of time” as used herein mean at least about 1 week, at least about 30 days, at least about six months, at least about one year, at least about two years, or at least about the time between preparation and use.

The term “active agent” as used herein means an agent that causes the desired therapeutic effect, such as antimicrobial activity.

The term “agent” as used herein indicates a compound or mixture of compounds that, when added to a composition or product, tend to produce a particular effect on the composition's or product's properties.

The term “alkali metal base” as used herein refers to a basic substance that comprises an inorganic anion and an alkali metal cation and that is suitably soluble in water or aqueous solutions, and includes, for example, sodium hydroxide, potassium hydroxide, lithium hydroxide, pentasodium triphosphate, potassium, pyrophosphate, sodium pyrophosphate, sodium carbonate, potassium carbonate, and lithium carbonate, and mixtures thereof.

The term “alkaline earth metal base” as used herein refers to a basic substance that comprises an inorganic anion and an alkaline earth metal cation and that is suitably soluble in water or aqueous solutions, and includes, for example, barium hydroxide, calcium hydroxide, magnesium hydroxide and calcium hydroxide, and mixtures thereof.

The term “alkylamine” refers to a basic compound of the general formula RR′R″N, wherein R, R′ and R″ are the same or different and represent an organic alkyl group that is optionally substituted or H, and that is suitably soluble in water or aqueous solutions. Examples of such compounds include, triethylamine, trimethylamine, diisopropylamine, and the like.

The term “antimicrobially effective amount” as used herein means an amount sufficient to achieve a desired antimicrobial result, which amount may be determined by a person skilled in the art.

The term “aqueous solution” as used herein means a solution wherein the solvent is substantially water, although small amounts, for example, less than 10, 9, 8, 7, 6, 5, 4, 3, 2 or 1% (v/v) of a non-aqueous solvent may be present.

The term “base” as used herein refers to a substance that can accept hydrogen ions (protons), or more generally donate electron pairs.

The term “chlorite” as used herein refers to the anion “ClO₂⁻”. Anionic species typically exist in aqueous solutions in dissociated form, however the anion is derived from a parent salt containing an anion and a cation.

The term “composition” as used herein refers to a mixture comprising two or more substances.

In understanding the scope of the present disclosure, the term “comprising” and its derivatives, as used herein, are intended to be open ended terms that specify the presence of the stated features, elements, components, groups, integers, and/or steps, but do not exclude the presence of other unstated features, elements, components, groups, integers and/or steps. The foregoing also applies to words having similar meanings such as the terms, “including”, “having” and their derivatives. The term “consisting” and its derivatives, as used herein, are intended to be closed terms that specify the presence of the stated features, elements, components, groups, integers, and/or steps, but exclude the presence of other unstated features, elements, components, groups, integers and/or steps. The term “consisting essentially of”, as used herein, is intended to specify the presence of the stated features, elements, components, groups, integers, and/or steps as well as those that do not materially affect the basic and novel characteristic(s) of features, elements, components, groups, integers, and/or steps.

The term “dressing” as used herein refers to an adjunct used for application to a wound to promote healing and/or prevent further harm. A dressing is designed to be in direct contact with the wound or wound bed. Dressings may also be referred to in the art as films, gels, foams, pads, bandages, sponges, gauzes, hydrogels, alginates, plasters, compresses, and the like.

The term “effective amount” as used herein means an amount sufficient to achieve the desired result and accordingly will depend on the ingredient and its desired result. Nonetheless, once the desired effect is known, determining the effective amount is within the skill of a person skilled in the art.

In general, the “error bars” on the graphs represent the standard error of the mean value, whereas the top of the solid, shaded bar represents a single data value, which is the mean value of the distribution of data values.

When used with respect to methods of treatment and the uses of compositions of the application, a subject “in need thereof” is a subject who has been diagnosed with, suspected of having, susceptible to or previously treated for the condition to be treated.

The term “inorganic base” as used herein refers to a basic substance that is not hydrocarbon based. The inorganic base is suitably one that is soluble in water or aqueous solutions, is compatible with the other ingredients in the dressing materials of the application and is capable of maintaining the pH of the absorbent material at a value greater than or equal to about 10. Examples of inorganic bases include, for example, alkali metal bases, alkaline earth metal bases, ammonia and ammonia hydroxides, and mixtures thereof.

The term “isotonic” as used herein means having the same salt or solute concentration as the normal cells of the body and body fluids.

The term “organic base” as used herein refers to a basic substance that is hydrocarbon based. The organic base is suitably one that is soluble in water or aqueous solutions, is compatible with the other ingredients in the dressing materials of the application and is capable of maintaining the pH of the absorbent material at a value greater than or equal to about 10. Examples of organic based include, for example, alkylamines, alkylamides, methyloxides and citrates, and mixtures thereof.

“Pharmaceutical composition” refers to a composition of matter for pharmaceutical use. The terms “pharmaceutical composition” and “formulation” are used interchangeably.

The term “pharmaceutically acceptable” means compatible with the treatment of animals, in particular, humans.

“Published material” means a medium providing information, including printed, audio, visual, or electronic medium, for example a flyer, an advertisement, a product insert, printed labeling, an internet web site, an internet web page, an internet pop-up window, a radio or television broadcast, a compact disk, a DVD, a podcast, an audio recording, or other recording or electronic medium.

The term “solution” as used herein refers to a composition in liquid state comprising at least one solute dissolved in a suitable solvent.

The term “stabilized chlorite” as used herein refers to a composition or substance, comprising chlorite ions (ClO₂⁻) and in which the concentration of chlorite ions, the pH and/or the activity remains stable for an acceptable period of time prior to use. In a stabilized chlorite, the chlorite ions do not substantially degrade and the activity of the chlorite ions is substantially maintained prior to use. The stabilized chlorite may contain a buffer, such as a sodium carbonate/sodium hydroxide buffer system, which maintains the alkaline pH of the formulation. The concentration of chlorite ions may be monitored, for example, by high performance liquid chromatography (HPLC).

The term “subject” as used herein includes all members of the animal kingdom, including mammals, and suitably refers to humans.

Terms of degree such as “substantially”, “about” and “approximately” as used herein mean a reasonable amount of deviation of the modified term such that the end result is not significantly changed. These terms of degree should be construed as including a deviation of at least ±5% of the modified term if this deviation would not negate the meaning of the word it modifies.

“Topical” as used herein includes topical application to the skin, nail, mucosa, or wound thereof. Topical use may, for example, be for conferring a therapeutic or cosmetic benefit to its user. Specific topical uses include, for example, local or regional application of substances.

The term “topical administration” is used herein to include the delivery of a substance, such as an active agent, to the skin, nail, mucosa, or wound thereof.

The term “treating” or “treatment” as used herein and as is well understood in the art, means an approach for obtaining beneficial or desired results, including clinical results. Beneficial or desired results can include, but are not limited to, alleviation or amelioration of one or more symptoms or conditions, diminishment of extent of disease, stabilizing (i.e. not worsening) the state of disease, prevention of disease spread, delaying or slowing of disease progression, amelioration or palliation of the disease state, diminishment of the reoccurrence of disease, and remission (whether partial or total), whether detectable or undetectable. “Treating” and “Treatment” can also mean prolonging survival as compared to expected survival if not receiving treatment. Treatment methods may comprise administering to a subject an antimicrobially effective amount of an active agent and optionally consists of a single administration, or alternatively comprises a series of applications. The length of the treatment period depends on a variety of factors, such as the severity of the condition, the age of the patient, the concentration of active ingredient or agent, the activity of the active agent, and/or a combination thereof. It will also be appreciated that the effective dosage of the agent used for the treatment may increase or decrease over the course of a particular treatment regime. Changes in dosage may result and become apparent by standard diagnostic assays known in the art. In some instances, chronic administration may be required. For example, the administration to the subject is in an amount and for a duration sufficient to treat the patient.

The term “treating wounds” or “wound healing” as used herein means to facilitate the contraction, closure and faster healing of wounds using the materials of the present application, for example, through their antimicrobial effect and by creating an environment conducive to wound healing. In particular the treatment of wounds or wound healing is facilitated as compared to wounds treated in an identical fashion except in the absence of the materials of the present application. Wound healing, for example, includes pressure, burn, post-operative or post-traumatic wound healing, or chronic wound healing as in the healing of diabetic ulcers, venous ulcers, arterial ulcers or decubitus ulcers.

The term “water” as used herein as an ingredient refers to pharmaceutically acceptable water.

The term “wound” as used herein refers to a type of injury or condition which includes, without limitation, infected wounds, pressure wounds, chronic wounds, delayed or problematic post-traumatic or post-op wound healing, decubitus ulcers, chronic leg ulcers in venous insufficiency, ulcers & wounds due to arterial blood flow, disorders or diabetic microangiopathy, diabetic ulcers, gangrene, psoriasis, atopic or neuro dematitis and burns. The term “wound” also includes full or partial thickness wounds, with light, moderate or heavy exudates as well as non-exudating wounds.

The term “w/v” as used herein means the number of grams of solute in 100 mL of solution.

The term “w/w” as used herein means the number of grams of solute in 100 g of solution.

### II. Wound Dressings

The wound dressings of the present application are intended for use in or on wounds to locally promote wound healing. It has surprisingly been found that a chlorite-containing wound dressing can be made by heating an absorbent material with a base to adjust its pH so that it is greater than or equal to about 10, prior to impregnation with the chlorite. Accordingly, in one embodiment of the application, there is included a wound dressing comprising an absorbent material, an effective amount of chlorite and an amount of a base to provide a pH of the absorbent material greater than or equal to about 10.

Without wishing to be limited by theory, one beneficial effect of the materials of the present application is due to their antimicrobial properties. It is known that wound dressings coated or impregnated with antiseptic agents, such as silver sulphadiazine or silver nitrate, are useful in wound healing. However, strong cytotoxic effects have been associated with many of these agents (Teepe et al., J. of Trauma 35(1):8-19, 1993). By contrast, wound dressings impregnated with chlorite possess antimicrobial properties at concentrations well below the onset of cytotoxicity of other known antiseptic agents. In one embodiment of the application, the impregnated absorbent materials of the present application are effective upon application (i.e. do not require activation in situ or after application), stable without lyophilization, and have chlorite ion release behaviors that are substantially linear over a period of about 6 hr. These properties are conducive to creating an environment that is effective for wound healing while minimizing complicated procedures and cytotoxic effects.

Accordingly, in one embodiment of the invention, there is included a wound dressing having an active release behavior for chlorite ions that is substantially linear. In an embodiment, the release behavior is substantially linear for at least about 2 hours, at least about 4 hours, or at least about 6 hours. In a further embodiment, about 0.1% to about 10% of the total chlorite is released from the dressing after about 1 hour. In a further embodiment, about 5% to about 20% of the total chlorite is released from the dressing after about 6 hours following application to a subject.

In another embodiment, there is included a wound dressing having antimicrobial properties that are effective in creating an environment conducive to wound healing. In another embodiment, the present application includes a wound dressing comprising a chlorite ion concentration that decomposes by less than about 5% over the course of at least about 6 months at about room temperature. In another embodiment, the rate of decomposition is less than about 4.5%, 4%, 3.5%, 3.0%, 2.5%, 2%, 1.5%, 1.0%, or less than about 0.5%, and all fractions in between, over the course of at least six months at room temperature.

Advantageously, in the wound dressings of the present application the active agent is present in only a single layer of absorbent material. Other prior art dressings require at least two agents in two separate layers, which, when combined, form an active agent in situ. In situ generation of the active agent is not required with the dressings of the present application since the dressings possess the desired stability and maintain the activity of the chlorite active agent for an acceptable period of time. Therefore manufacture and use of the dressings of the present application is much simpler than prior art dressings. Accordingly, in a further embodiment of the application, there is included a wound dressing comprising a single active agent-containing absorbent layer.

### A. Chlorite

The wound dressings of the present application comprise chlorite. In an embodiment, the chlorite is present in an antimicrobially effective amount.

In another embodiment, the chlorite is added to the absorbent material as a stabilized chlorite composition. Non-limiting examples of stabilized chlorite-based compositions include those described in U.S. Pat. Nos. 6,350,438, 6,251,372, 6,235,269, 6,132,702, 6,077,502 and 4,574,084, the contents of each of which are incorporated by reference in their entirety.

In another embodiment, the stabilized chlorite composition is OXO-K993, or a composition comprising about 0.01-0.1%, 0.1%-1%, 1-10%, 10-20%, 20-30%, 30-50% or 50-90% (w/v) OXO-K993. In a further embodiment, the stabilized chlorite composition is a composition comprising WF10.

In an embodiment, the stabilized chlorite composition is a composition comprising about 2% (w/v) OXO-K993. In a further embodiment, the stabilized chlorite composition is a composition comprising about 2% (w/v) OXO-K993, about 2% (w/v) glycerol and about 96% (w/v) water. Such compositions are sold commercially under the names of Oxovasin™ and Oxoferin™ (Nuvo Manufacturing, Wanzleben, Germany), where 1 ml of Oxovasin™ comprises about 0.85 mg (or about 0.085% w/v) of chlorite in 1.0 ml water. The pH of Oxovasin™ is between 10.75 and 11.90. Therefore, in a further embodiment of the present application, the stabilized chlorite composition is a composition comprising Oxovasin™.

In an embodiment of the application, OXO-K993 is prepared using the following method:

Sodium chlorite (NaClO₂) and sodium hypochlorite (NaOCl) are mixed in a molar ratio of 4.8 to 1 in Water for Injection (WFI). The pH of the solution should be greater than pH 11.0. After addition of the catalyst, chlorylsulfuric acid [ClO₂⁺] [HSO₄⁻], to this mixture the following reaction can be observed:

2ClO₂+OCl⁻+2H⁺→2ClO₂+Cl⁻+H₂O (1)

The pH of the solution decreases. A portion of the chlorite is oxidized to chlorine dioxide (ClO₂) in the redox process described by Equation (1). In an equilibrium reaction, the developing chlorine dioxide forms an intense brown charge-transfer complex with the excess unoxidized chlorite, as shown in Equation (2):

ClO₂+ClO₂⁻ [symbol] [Cl₂O₄]⁻ (2)

9.65 mmol (per kg of the reaction solution) of sodium carbonate peroxohydrate (2 Na₂CO₃.3H₂O₂) is then added to the solution. Upon addition of sodium carbonate peroxohydrate, part of the chlorine dioxide is reduced back to chlorite, and oxygen is formed simultaneously:

2ClO₂+H₂O₂+2OH⁻→2ClO₂⁻+O₂+2H₂O (3)

After a suitable time, for example 15 minutes, 102 mmol (per kg of the reaction solution) of sodium peroxide (Na₂O₂) is added to the solution, which becomes completely decolorized as the remaining chlorine dioxide is reduced completely to chlorite. From sodium peroxide, oxygen evolves in a slow process that typically requires at least 4 weeks (Equation 4). Simultaneously, hydroxyl ions are formed, resulting in a high pH value (pH>13) of the solution, which thereby stabilizes the active substance chlorite.

2O₂²⁻+2H₂O→O₂+4OH⁻ (4)

The final reaction product, OXO-K993, resulting from this synthesis is a stable aqueous solution, which contains the active substance, chlorite (about 4.25%), together with the anions chloride (about 2.0%), chlorate (about 1.5%), and sulfate (about 0.7%), and sodium as the cation as well as a sodium carbonate/sodium hydroxide buffer system which maintains the alkaline pH of the formulation.

The skilled artisan will recognize that chlorite containing compositions, including derivatives of OXO-K993, WF10, Oxoferin™, Oxovasin™ or other chlorite-based solutions and their derivatives, are well within the scope of the application. The skilled artisan will also recognize that the chlorite compositions can be sterilized prior to use.

OXO-K993 and its derivatives (WF10, Oxoferin™, Oxovasin™) are also examples of solutions comprising a mixture of ions, since they comprise a combination of chlorite, chloride, chlorate, sulfate and sodium ions in an aqueous solvent. In one embodiment of the application, a solution comprising a mixture of ions is a composition comprising at least two different types of anions (chlorate and chlorite). In another embodiment of the application, the solution may comprise at least three different types of anions. In a further embodiment of the application, the solution may comprise at least four different types of anions. In an embodiment the chlorite is added to the absorbent material as a stabilized chlorite solution comprising, consisting of or consisting essentially of chlorite, chlorate, chloride, sulfate and sodium ions.

In a further embodiment of the present application, the chlorite is added to the absorbent material as a solution comprising chlorite ions prepared by dissolving a suitable chlorite salt in a suitable solvent, such as an aqueous solvent. The chlorite salt may be any suitable salt, including, for example, sodium chlorite, potassium chlorite, magnesium chlorite and barium chlorite. In an embodiment, the chlorite salt is sodium chlorite. In an embodiment the chlorite is a chlorite solution comprising, consisting of or consisting essentially of sodium chlorite. In a further embodiment, the sodium chlorite is obtained from a commercial source or is prepared using known processes. Any concentration of chlorite solution may be used.

Any composition of matter containing chlorite ions must also have at least one counter ion to maintain charge neutrality. Thus, according to one embodiment of the present application, the wound dressings comprise one or more cations. Non-limiting examples of possible cations include alkali metal cations (such as sodium or Na⁺) and alkaline earth cations. In another embodiment, the wound dressings comprising chlorite, further comprise sodium and/or potassium counter ions.

Other prior art dressings are applied dry and require wound exudate in order to provide a therapeutic effect. Applying a dry dressing to a wound, however, creates an osmotic gradient that drives fluid from the cells of the wound site into the dressing. For cells that are involved in wound repair or healing, an osmotic gradient is detrimental since the cells become deprived of internal fluids. Thus, according to one embodiment of the present application, there is provided a wound dressing that is isotonic with a subject's body fluids. In a further embodiment, the dressings of the present application are gentler on the wound site than dressings of the prior art. Determining isotonicity in this respect is well within the knowledge of the skilled artisan.

For application of the chlorite to the absorbent material, it is most convenient for the chlorite to be in solution. The solvent used to dissolve the chlorite in the preparation of a solution may be any suitable solvent in which a desired or effective amount of chlorite will dissolve and which is compatible with the absorbent material. In an embodiment, the solvent comprises, consists of or consists essentially of water. Other solvents may be combined with the water, such as certain alcohols (e.g. ethanol or isopropanol). When other solvents are present, it is an embodiment that they are present in an amount less than 50%, 40%, 30%, 20%, 10%, 5% or 1% (v/v), or fractions in between.

The amount of the chlorite in the dressings of the present application can vary depending on the intended use. In an embodiment, the amount of the chlorite is an amount effective to treat a wound based on the size and release rate of the dressing. This amount can be determined by a person skilled in the art. In an embodiment of the application, the chlorite is present in the wound dressings in an amount resulting from the absorption, by the absorbent material, of about 0.001, 0.005, 0.01, 0.05, 0.1, 0.5, 1, 2, 3, 4, 5, 6, 7, 8, 9, 10, 11, 12, 13, 14, 15, 16, 17, 18, 19 or 20, or fractions in between, times the dry weight of the absorbent material of the chlorite composition. In a further embodiment, the chlorite is present in the wound dressings in an amount resulting from the absorption, by the absorbent material, of about 5, 6, 7, 8, 9, 10, 11, 12, 13, 14 or 15, or fractions in between, times the dry weight of the absorbent material of the chlorite composition. In a further embodiment, the chlorite is present in the wound dressings in an amount resulting from the absorption, by the absorbent material, of about 10 times the dry weight of the absorbent material of the chlorite composition. By “dry weight” of the absorbent material, it is meant the weight of the absorbent material prior to washing or pretreatment.

Alternately, the amount of chlorite present in the absorbent material is an amount based on the percentage of chlorite ions. For example, in one embodiment of the application, the amount of chlorite present in the absorbent material is an amount having the equivalent percentage of chlorite ions contained in about 5 ml of tetrachlorodecaoxygen anion complex (TCDO), as described in Hinz et al. (The Lancet (1986)). In another embodiment, the percentage of chlorite ions in the absorbent material is equivalent to that contained in about 0.01 ml to about 0.1 ml, about 0.05 ml to about 0.15 ml, about 0.15 ml to about 1.0 ml, about 1.0 ml to about 1.5 ml, about 1.5 ml to about 2.0 ml, about 2.0 ml to about 2.5 ml, about 2.5 ml to about 3.0 ml, about 3.0 ml to about 3.5 ml, about 3.5 ml to about 4.0 ml, about 4.0 ml to about 4.5 ml or about 4.5 ml to about 5.0 ml of OXO-K993. In a further embodiment, the percentage of chlorite ions in the absorbent material is equivalent to that contained in about 0.01 ml to about 1.0 ml of OXO-K993.

### B. Absorbent Material

The absorbent material can be formed from any suitable material including, foams and woven and non-woven fabrics and combinations thereof. Typically, the material comprises a soft, flexible, porous-type material, suitable for use in contact with wounds and use as a wound dressing. The material can be natural or synthetic, or a combination thereof. Natural materials include, for example, collagen sponge, cellulose, cottons and open cell foam materials. Synthetic materials include, for example, urethane-type foams, rayons, polyesters, and membrane materials. The material may also be a combination of natural and synthetic materials. In an embodiment, the absorbent material is a woven material or gauze. In another embodiment, the absorbent material is selected from films, gels, foams, hydrocolloids, alginates, hydrogels, polysaccharide pastes, granules and beads. In a further embodiment the absorbent material is a multilayer laminate material containing a combination of various types of materials, at least one layer of which is absorbent. In a further embodiment, the absorbent material is a polyurethane-based dressing material, for example, Medisponge™ materials available from Lendell Manufacturing Inc. St. Charles, Mich., USA, Rynel™ Foams available from Rynel, Inc. Wiscasset, Me., USA, or Suprasorb™ materials available from the Lohmann-Rauscher Group. In a further embodiment multilayer laminate material is an engineered composite of non-adherent and non-limiting structures, created by laminating multiple layers of nets, nonwovens or textiles to create complex materials, such as Stratex™ materials from Delstar Technologies, Middletown, Del., USA.

For pharmaceutical use, the absorbent material is optionally sterilized by any known sterilization technique, for example, ethylene oxide treatment, ionizing radiation (e.g. gamma radiation treatment), heat or by aseptic manufacturing.

The absorbent material may be suitably sized to fit a predetermined wound type and, without limitation, may be round, elliptical, square, rectangular, polygon, polygon with rounded corners or three dimensional (e.g. spherical balls). Typically, the absorbent material ranges from about 0.1 mm to about 10 cm in thickness and has a dimension in the range of from about 1×1 cm square to about 20×20 cm square. The absorbent material may also be suitably thick and have an areal dimension that provides an absorptive capacity for handling wound exudate. In addition, the absorbent material may be fully or less than fully (i.e. partially) saturated with chlorite depending on the type of wound being treated (e.g. moderate to heavy exudate wound).

In an embodiment of the application, the absorbent material further comprises means for retaining the dressing on or in a wound, for example, it may comprise adhesive portions or an attachable backing. Alternately, a secondary bandage may be placed over the dressing to retain the dressing in place and/or to prevent run-off. In another embodiment, the absorbent material further comprises additional layers, for example, a layer of nonstick film over the absorbent material to prevent the wound from adhering to the dressing and/or an outer moisture repellant layer to prevent the permeation of moisture into the material or wound after it is applied.

### C. Base

Absorbent materials of the present application, impregnated with chlorite, comprise a pH of greater than or equal to about 10. Studies have demonstrated that impregnating an absorbent material with a chlorite solution alone or following pre-soaking with water, is not effective in maintaining a pH of greater than or equal to about 10. Also, it has surprisingly been found that weak bases, such as phosphate buffered saline (PBS), are not effective in stabilizing an absorbent material dosed with a chlorite solution. These weak bases are incapable of maintaining a pH of greater than or equal to about 10, resulting in a concomitant decrease in the stability of the resulting dressing (see examples).

Suitable bases of the application include, for example, inorganic bases, such as alkali metal bases, alkaline earth metal bases, ammonia and ammonia hydroxides, and mixtures thereof, and organic bases, such as alkylamines, alkylamides, methyloxides and citrates, and mixtures thereof, or a mixture of an inorganic and organic base. These bases are those capable of stabilizing the absorbent material at a pH of greater than or equal to about 10. In an embodiment of the application, the base is an inorganic base, such as an alkali metal base. In one embodiment, the alkali metal base is sodium carbonate, pentasodium triphosphate, potassium, pyrophosphate, sodium pyrophosphate or potassium carbonate, or a mixture thereof. In another embodiment, the alkali metal base comprises, consists of or consists essentially of sodium carbonate or potassium carbonate.

In an embodiment the base is added to the absorbent material as a solution with a concentration that does not decompose the absorbent material or chlorite ion concentration, for example, a concentration of about 0.01, 0.05, 0.10, 0.15, 0.20, 0.25, 0.30, 0.35, 0.40, 0.45 or 0.50 M (mol/L), or fractions in between. In a further embodiment the base is added to the absorbent material as a solution having a concentration of about 0.01, 0.05, 0.10, 0.15 or 0.20 M, or fractions in between. In another embodiment the base is added to the absorbent material as a solution having a concentration of about 0.10 M.

In a further embodiment, the absorbent material is treated with an excess amount of a base solution and the excess of the base solution is removed by squeezing and/or drying, for example, prior to treating the absorbent material with the chlorite.

### D. Color Stabilizing Agent

In addition to their antimicrobial properties, chlorite is expected to have whitening qualities. Sodium chlorite, a known bleaching agent, is used to effectively bleach textiles such as cotton, bast fibers, and man-made fibers like nylon, Perlon, Dralon, and Rhovyl. It is surprising then, that absorbent materials of the present application impregnated with a chlorite, undergo discoloration. More surprising is the fact that pretreatment of the absorbent material with arginine results in prevention of this discoloration (see examples).

Thus, in accordance with one embodiment of the application, the absorbent material further comprises a color stabilizing agent, such as, arginine. In a further embodiment the color stabilizing agent is added to the absorbent material in an effective amount, for example, as a solution having a concentration of about 0.01, 0.05, 0.10, 0.15, 0.20, 0.25, 0.30, 0.35, 0.40, 0.45 or 0.50 M (mol/L), or fractions in between. In another embodiment, the color stabilizing agent is added to the absorbent material as a solution having a concentration of about 0.01, 0.05, 0.10, 0.15 or 0.20 M, or fractions in between. In a further embodiment, the color stabilizing agent is added to the absorbent material as a solution having a concentration of about 0.10 M.

In another embodiment, the absorbent material is treated with an excess amount of the color stabilizing agent, before, after or concurrently with the base, and the excess of the color stabilizing agent is removed by squeezing and/or drying, for example, prior to treating the absorbent material with the chlorite. In a further embodiment, a color stabilizing agent, such as arginine, is used to prevent discoloration of a material comprising chlorite. In still a further embodiment, a color stabilizing agent, such as arginine, is used to stabilize or prevent decomposition of the absorbent material or chlorite ion concentration in the dressing.

The present application also includes a use of arginine as a color stabilizing agent in a wound dressing, the wound dressing comprising an absorbent material, an effective amount of chlorite and an amount of a base to provide a pH of the absorbent material greater than or equal to about 10. The present application also includes a method of stabilizing the color of a wound dressing, the wound dressing comprising an absorbent material, an effective amount of chlorite and an amount of a base to provide a pH of the absorbent material greater than or equal to about 10, the method comprising applying arginine to the wound dressing. By “stabilizing the color” it is meant inhibiting discoloration of the initial color of the dressing.

### E. Other Components

In further embodiments of the present application, the wound dressings further comprise other additives or agents that are desired for particular applications. Such additives or agents include, but are not limited to, anti-oxidants, humectants, solvents, antibiotics, antimicrobial agents (e.g. silver or silver compounds, iodine and chlorohexidine), dyes, perfumes, fragrances and the like, and mixtures thereof.

### III. Methods and Uses of the Application

The present application also includes a method for preparing the wound dressings described hereinabove comprising:
(a) treating an absorbent material with a base solution to provide a pH that is greater than or equal to about 10; and
(b) treating the absorbent material with an effective amount of chlorite,
wherein the absorbent material is treated with the chlorite concurrently or after treatment with the base.

In an embodiment, the absorbent material is treated with the base by soaking the material with a solution comprising the base, for example an aqueous solution comprising the base. In an embodiment the absorbent material is treated with the base at a temperature of about 0° C. to about 40° C., about 5° C. to about 35° C. or about 10° C. to about 25° C.

In another embodiment, the absorbent material is treated with a base to provide a pH of the absorbent material of about 10, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9 or 13.

In another embodiment of the application, the absorbent material is first treated with a base solution to provide a pH that is greater than or equal to about 10, followed by removing excess base solution and/or drying the absorbent material and impregnating the absorbent material with an effective amount of chlorite.

In a specific embodiment of the application, the absorbent material is treated with an excess amount of 0.1 M sodium carbonate at room temperature followed by either squeezing excess base solution from the material (e.g. using a hand roller) and/or drying (e.g. at 45° C. in an incubator overnight).

In another embodiment of the application, the absorbent material is treated with the base and chlorite concurrently. For example, the base and chlorite solutions may be combined prior to treatment of the absorbent material, or alternatively, the base and chlorite solutions are added to the absorbent material simultaneously, or any other suitable methods for impregnating the absorbent material with the chlorite and base.

By treating the absorbent material, it is meant that the material is contacted with the base and chlorite in such a way that the base and chlorite are absorbed into the absorbent material. This may be done by immersion, spraying or any other suitable method. Following treatment, excess base solution and chlorite are removed, for example by squeezing and/or drying.

In an embodiment of the application, the absorbent material is also treated with a color stabilizing agent, such as arginine. In an embodiment, the color stabilizing agent is added to the absorbent material in the same solution as the base and/or chlorite. In a further embodiment, the color stabilizing agent is added to the absorbent material in a separate solution from the base, either before, after or concurrently with the base.

In an embodiment of the application, the absorbent material is also treated with additives or agents that are desired for particular applications. Such additives or agents include, but are not limited to, anti-oxidants, humectants, solvents, antibiotics, antimicrobial agents (e.g. silver or silver compounds, iodine, and chlorohexidine), dyes, perfumes, fragrances and the like, and mixtures thereof. These additives or agents can be added at any suitable time in the method of making the wound dressings of the application.

In an embodiment of the application, the absorbent materials, either dried or wet, are impregnated with the effective amount of the chlorite by immersion in a chlorite composition until maximum amounts of the composition are absorbed. In an embodiment, the absorbent materials absorb about 0.001, 0.005, 0.01, 0.05, 0.1, 0.5, 1, 2, 3, 4, 5, 6, 7, 8, 9, 10, 11, 12, 13, 14, 15, 16, 17, 18, 19 or 20, or fractions in between, times its dry weight of the chlorite composition. In a further embodiment, the absorbent materials absorb about 5, 6, 7, 8, 9, 10, 11, 12, 13, 14 or 15, or fractions in between, times its dry weight of the chlorite composition. In a further embodiment, the absorbent material absorbs about 10 times its dry weight of the chlorite solution. By “dry weight” of the absorbent material, it is meant the weight of the absorbent material prior to washing or pretreatment.

Alternately, the absorbent materials, either dried or wet, are impregnated with an effective amount of the chlorite composition based on the percentage of chlorite ions. For example, in one embodiment of the application, the amount of chlorite absorbed by the material is an amount having the equivalent percentage of chlorite ions contained in about 5 ml of tetrachlorodecaoxygen anion complex (TCDO), as described in Hinz et al. (The Lancet (1986)). In another embodiment, the amount of chlorite absorbed by the material is an amount having the equivalent percentage of chlorite ions contained in about 0.0 ml to about 1 ml, about 1 ml to about 5 ml, about 5 ml to about 10 ml, about 10 ml to about 15 ml, about 15 ml to about 20 ml, about 20 ml to about 25 ml, about 25 ml to about 30 ml, about 30 ml to about 35 ml, about 35 ml to about 40 ml, about 40 ml to about 45 ml or about 45 ml to about 50 ml of TCDO. In a further embodiment, the amount of chlorite absorbed by the material is an amount having the equivalent percentage of chlorite ions contained in about 5 to about 10 ml of TCDO.

In one embodiment of the application, the impregnated absorbent materials of the present application are effective upon application (i.e. do not require activation in situ or after application), stable without lyophilization, and have chlorite ion release behaviors that are substantially linear over a period of about 6 hr. In another embodiment, the wet wound dressing is isotonic with a subject's body fluids.

In an embodiment, the method of preparing a wound dressing further comprises sealing the dressing in a sealable enclosure. In an embodiment, the wound dressing is moist or wet when sealed in the sealable enclosure and/or when used. In another embodiment, the wound dressing is dried prior to being sealed in the sealable enclosure and/or prior to use. In yet another embodiment, the wound dressing is not lyophilized prior to being sealed in the sealable enclosure and/or prior to use.

The present application further includes a wound dressing prepared by a process comprising:
(a) treating an absorbent material with a base solution to provide a pH that is greater than or equal to about 10; and
(b) treating the absorbent material with an effective amount of chlorite,
wherein the absorbent material is treated with the chlorite concurrently or after treatment with the base.

It has surprisingly been found that, in the absence of lyophilization, a wound dressing comprising chlorite can be manufactured and remains stable for an acceptable period of time. In particular, the dressing is stabilized by maintaining a pH greater than or equal to about 10, thereby preventing decomposition of the chlorite ions.

In an embodiment, the resulting chlorite-containing dressings are kept at a temperature of about 0° C. to about 30° C. In another embodiment, the dressings are freeze-dried. In another embodiment, the dressings are packaged in a compatible wrapping or other closure system. In another embodiment, the wound dressings do not allow microbial growth or proliferation once prepared.

In other embodiments of the application the wound dressings are, if desired, packaged in a compatible wrapping or other closure system, for example a system approved by the Food and Drug Administration (FDA) or other regulatory body, which contain one or more units of the would dressing. In an embodiment, the packaging or wrapping is also accompanied by any notices prescribed by a governmental agency regulating the manufacture, use, or sale of such products.

Accordingly, in a further embodiment of the application, there is included a pharmaceutical package comprising:
(a) a wound dressing comprising an absorbent material, an effective amount of chlorite and an amount of a base to provide a pH of the absorbent material greater than or equal to about 10;
(b) a sealable enclosure for the wound dressing; and
(c) optionally, instructions for use.

In an embodiment, the pharmaceutical package comprises a single active agent-containing absorbent layer, wherein the active agent is chlorite.

In another embodiment, the wound dressing is moist or wet when placed into the sealable enclosure and is sealed into the enclosure in this state. It is a further embodiment that the wound dressing loses less than 40%, less than 30%, less than 20%, less than 10%, less than 5%, less than 2%, or less than 0.5% of its weight over an acceptable period of time while sealed in the enclosure. In another embodiment, the moist or wet wound dressing is isotonic with a subject's body fluids. In a further embodiment of the application, the impregnated absorbent materials of the present application are effective upon application (i.e. do not require activation in situ or after application), stable without lyophilization, and have chlorite ion release behaviors that are substantially linear over a period of about 6 hr.

In another embodiment, the wound dressing is dried before being placed into the sealable enclosure and is sealed into the enclosure in this state.

The sealable enclosure is made from any suitable material that is compatible with the wound dressings of the application. Desirably, the material is impermeable and inert to the aqueous solutions that are present in and on the wound dressing and is capable of sterilization and maintaining a sterile environment once the dressing is sealed into the enclosure. By “sealed into the enclosure” it is meant that the dressing is enclosed such that substantially no substances, including air, may pass in or out of the enclosure. Examples of suitable materials for the sealable enclosure include, but are not limited to, laminate foils, such as low density polyethylene (LDPE) foils, and other materials that provide air- and liquid-tight enclosures that are resistant to oxidation under alkaline conditions. In an embodiment, the sealable enclosure is adapted to the shape of the wound dressing. In another embodiment, the sealable enclosure comprises means for opening the enclosure.

As noted above, the wound dressings of the present application require that the active agent be present in only a single layer of absorbent material. Other prior art dressings require at least two agents in two separate layers, which, when combined, form an active agent in situ. In situ generation of the active agent is not required with the dressings of the present application since the dressings possess the desired stability and main the activity of the chlorite active agent for an acceptable period of time. Therefore manufacture of the dressings of the present application is much simpler than prior art dressings.

In a further embodiment, the wound dressings, when used have an active release behavior for chlorite ions that is substantially linear. In an embodiment, the release behavior is substantially linear for at least about 2 hours, at least about 4 hours, or at least about 6 hours. In a further embodiment, about 0.1% to about 10% of the total chlorite is released from the dressing after about 1 hour. In a further embodiment, about 5% to about 20% of the total chlorite is released from the dressing after about 6 hours.

The wound dressings of the present application are novel, therefore the application further includes all uses of these dressings as well as methods which include these dressings. In a particular embodiment, there is included a use of the wound dressings of the present application as an antimicrobial agent.

The application further includes a method for treating wounds comprising applying a wound dressing of the application to a subject in need thereof. In one embodiment, the wound dressing is applied to the wound or wound bed of the subject.

The application further includes a use of a wound dressing of the application for wound healing, including pressure, burn, post-operative or post-traumatic wound healing, or chronic wound healing as in the healing of diabetic ulcers, venous ulcers, arterial ulcers or decubitus ulcers.

Advantageously, the wound dressings of the present application are useful in creating an environment conducive to the wound healing process. The wound dressings comprising chlorite have the desired antimicrobial properties effective for promoting wound healing.

The present application also includes a method for treating a condition comprising applying a wound dressing of the application to a subject in need thereof. In one embodiment, the wound dressing is applied to the wound or wound bed of the subject. In another embodiment, the dressing is applied to a cavity wound of the subject alone, or in combination with a bandage to prevent run-off and/or hold the dressing in place. The wound dressing are particularly useful for the treatment of any condition or injury for which topical administration of chlorite is beneficial, including creating an environment conducive to wound healing. Examples of such conditions or injuries include, but are not limited to, skin diseases and skin disorders (including topical and neuro dermatitis, psoriasis, herpes simplex, herpes zoster and acne), infections, burns and wound healing (including pressure, burn, post-operative and post-traumatic wound healing, as well as chronic wound healing in the case of diabetic ulcers, venous ulcers, arterial ulcers, decubitus ulcers and the like). The dressing is also suitable for use on full thickness wounds (e.g. Stage III or V ulcers) with light, moderate or heavy exudates as well as non exudating wounds.

Further included in the present application is a use of a wound dressing of the application as an antimicrobial agent.

In one embodiment, the treatment is administered at least once a week. In another embodiment, the treatment is administered at least twice a week. In still another embodiment, the treatment is administered at least three times a week. In yet another embodiment, the treatment is administered at least four times a week. In an even further embodiment, the treatment is administered as prescribed or until the condition has ameliorated to where further treatment is not necessary.

The dressings of the present application are useful and effective when applied topically to treat a condition. In one embodiment of the application, the amount of the active agent present in the dressing will be the amount that is antimicrobially effective, i.e., an amount that is effective in creating an environment conducive to wound healing. The antimicrobially effective amount will vary depending on the subject and the severity of the affliction and can be determined routinely by one of ordinary skill in the art. Exemplary dosing of a chlorite-containing solution for the treatment of ulcerative wounds, for example, is provided in Hinz et al., The Lancet (1986), the contents of which is incorporated by reference in its entirety.

In one embodiment, a method of using the wound dressings comprises informing a user of certain safety or clinical effects. For example, the user may be informed that the dressings are more stable, simple to use or therapeutically effective than other dressings that provide, or would be expected to provide, a similar therapeutic effect. The user may be informed by way of published material such as a label or product insert.

The following non-limiting examples are illustrative of the present application:

### IV. Examples

### Example 1

### General Procedure for Preparation of Dressing Materials

The materials were cut in approximately 2 cm×2 cm pieces and weighed. The materials are then pretreated with the base solution for about 10 minutes. The excess base solution was removed by squeezing (e.g. hand rolling) excess solution and/or by drying and the dressings were dosed with chlorite solution. All dressing materials absorbed approximately 10× their dry weight (prior to pretreatment) of the chlorite solution. The dressing materials were sealed in pouches made from a 4 layer laminate. Various tests were conducted, including pH, color and chlorite ion measurements.

### Example 2

### Evaluation of Pre-Treatment Processes

A list of the materials that were tested is found in Table 1A. Sources of the other ingredients used are listed in Table 1B. Various treatment conditions were evaluated for their effect on the pH and the amount of chlorite remaining in the sponge (measured using HPLC). Measurement of surface pH of the sponge with a pH meter equipped with a surface measuring probe (Fisher 02-226-9) were carried out. The treatment conditions that were evaluated were as follows:

1. Treatment with Carbonates:
(a) sodium carbonates
   (i) Na₂CO₃ treatment
   (ii) Na₂CO₃/arginine treatment
(b) potassium carbonates

2. Oxovasin™ Treatment Conditions:
(a) wet condition (sponge squeezed with a hand roller after treatment with base)
(b) dry condition (sponge squeezed with a hand roller and dried at 45° C. in an incubator overnight after treatment with base)

3. Concentrations of Carbonates
(a) 0.1 M Na₂CO₃
(b) 0.2 M Na₂CO₃
(c) 0.1 M Na₂CO₃/0.1 M arginine

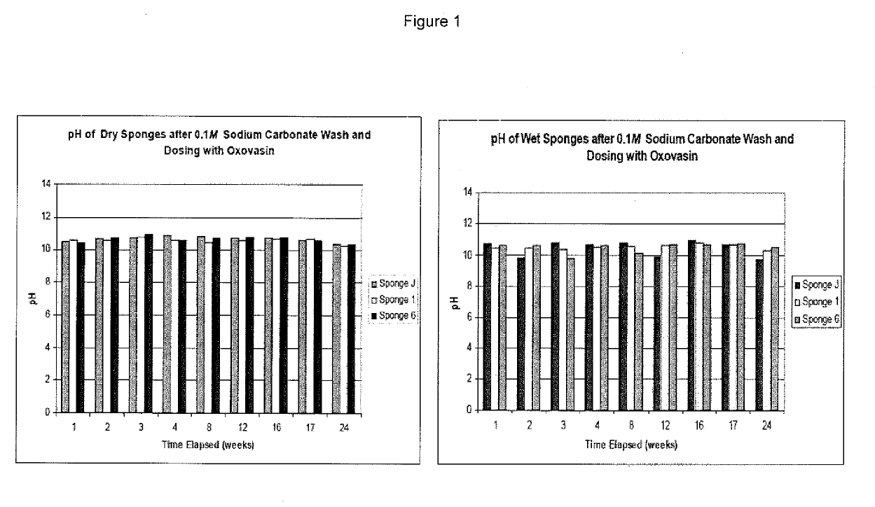

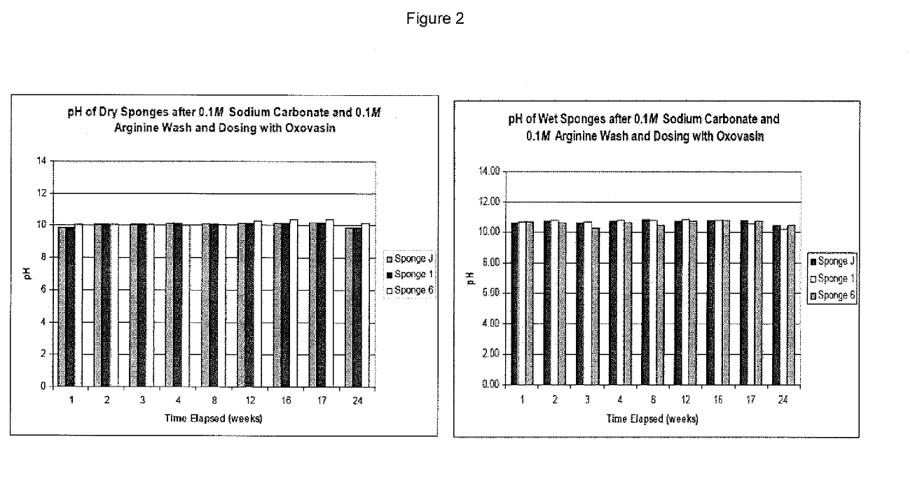

Representative results are shown in FIGS. 1 and 2 and in Table 2. FIG. 1 shows the effect of wet vs dry treatments on the pH as a function of time for absorbent materials 1, 6 and J (see Table 1A) that have been pre-treated with 0.1 M Na₂CO₃ and dosed with Oxovasin™. FIG. 2 shows the effect of wet vs dry treatments on the pH as a function of time for absorbent materials 1, 6 and J (see Table 1A) that have been pre-treated with 0.1 M Na₂CO₃/0.1 M arginine and dosed with Oxovasin™. Table 2 shows the percent chlorite remaining after 17 weeks, as determined by HPLC, in sponge 1 and 6 treated as described in FIGS. 1 and 2. The lower chlorite ion content for the wet treatments may be due to a dilution effect because of the presence of moisture in the foam. Dressings fabricated with absorbent material J were not analyzed due to the small amount of sample obtained.

The results from the evaluations performed in the present example have shown that the optimal results are obtained with sodium carbonate or sodium carbonate/arginine pretreatments.

### Example 3

### Further Studies

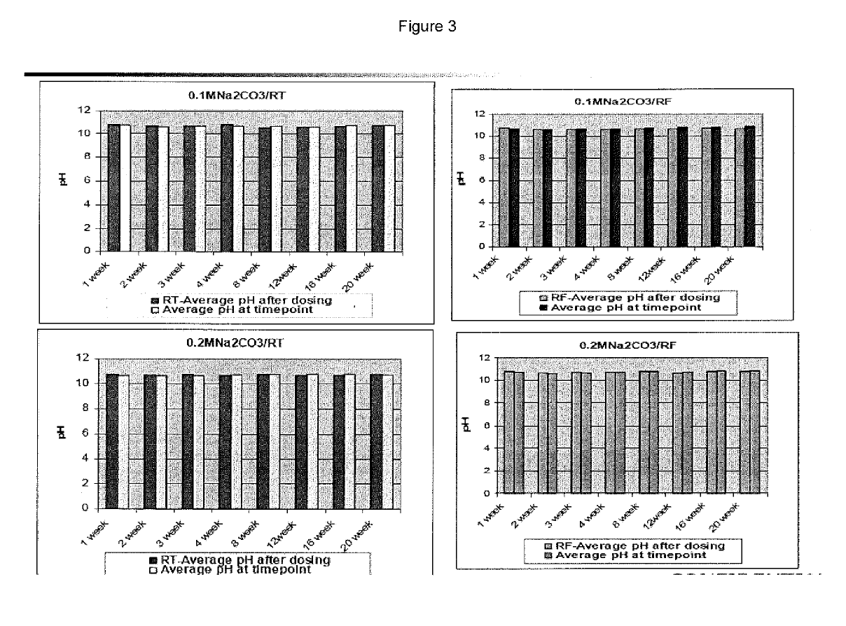

Further studies on the stability of sponges 1 and 6 from Table 1A, a Stratex Sponge 7.9NLYBE and some marketed dressings under various conditions were performed. The results are shown in FIGS. 3-7 and in Tables 3-5.

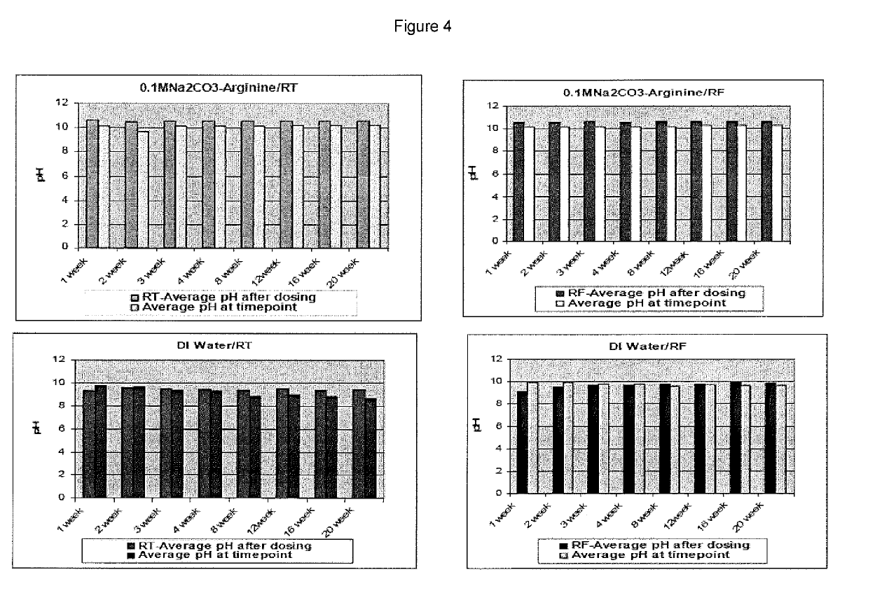

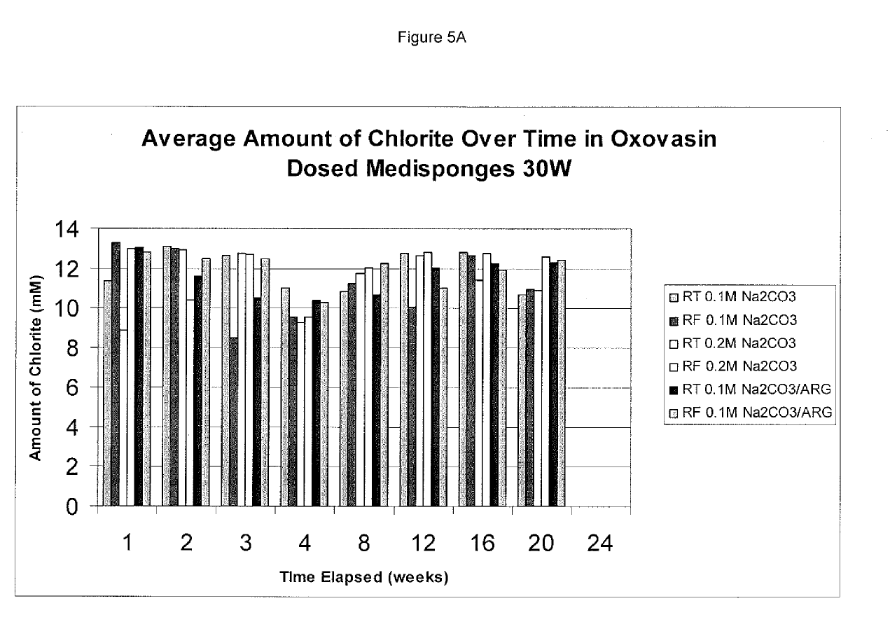

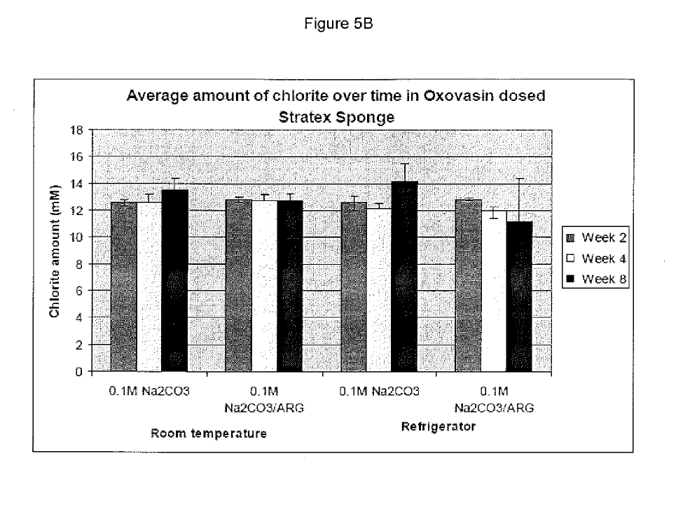

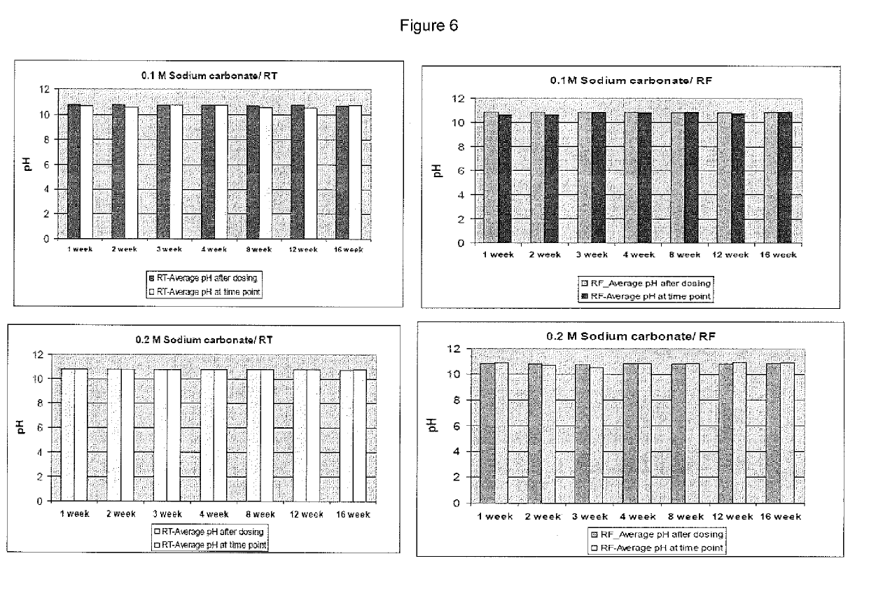

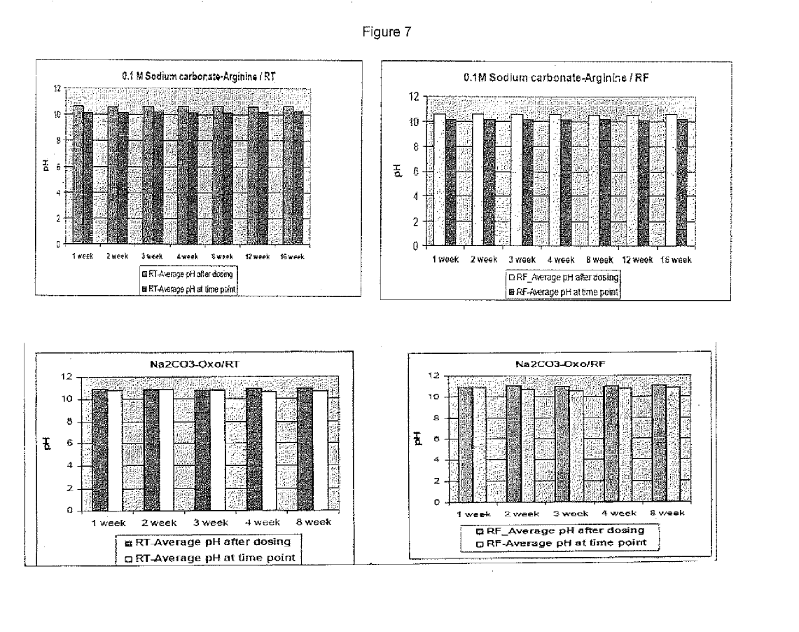

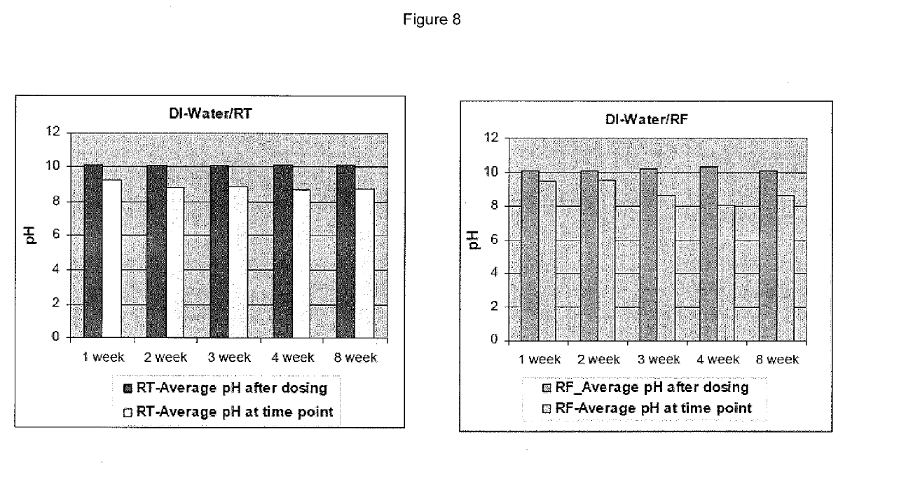

FIG. 3 shows the pH profile over 20 weeks of a Medisponge™ W30 pre-treated with either 0.1 M Na₂CO₃ or 0.2 M Na₂CO₃ and dosed with Oxovasin™ then kept at room temperature (RT) or at refrigerator temperature (RE). FIG. 4 shows the pH profile over 20 weeks of a Medisponge™ W30 pre-treated with either 0.1 M Na₂CO₃/arginine or deionized water (DI) and dosed with Oxovasin™ then kept at room temperature (RT) or at refrigerator temperature (RF). FIG. 5A-B show the average amount of chlorite (as determined using HPLC) over time in Oxovasin™ dosed Medisponge™ (FIG. 5A) and Stratex Sponge (FIG. 5B) that have been pre-treated using conditions from FIGS. 3 and 4. FIG. 6 shows the pH profile over 16 weeks of a Medisponge™ 50P pre-treated with either 0.1 M Na₂CO₃ or 0.2 M Na₂CO₃ and dosed with Oxovasin™ then kept at room temperature (RT) or at refrigerator temperature (RF). FIG. 7 shows the pH profile over a period of up to 16 weeks of a Medisponge™ 50P pre-treated with either 0.1 M Na₂CO₃/arginine or 0.1 M Na₂CO₃ and dosed with Oxovasin™ then kept at room temperature (RT) or at refrigerator temperature (RE). FIG. 8 shows the pH profile over 8 weeks of a Medisponge™ 50P pre-treated deionized water (DI) and dosed with Oxovasin™ then kept at room temperature (RT) or at refrigerator temperature (RF).

Table 3 presents the pH data after 2 weeks and 4 weeks for a Stratex sponge, pre-treated with either 0.1 M Na₂CO₃ or 0.1 M Na₂CO₃/arginine and kept at either room temperature or at refrigerator temperature. Table 4 presents the amount of chlorite present in a Stratex sponge (determined using HPLC) at 2 weeks and 4 weeks following pretreatment with either 0.1 M Na₂CO₃ or 0.1 M Na₂CO₃/arginine and kept at either room temperature or at refrigerator temperature.

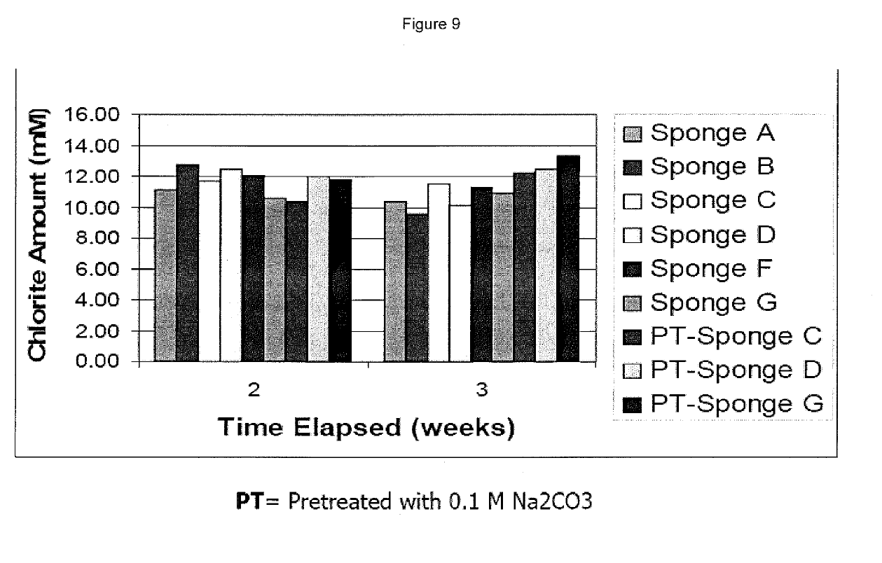

The details of the various marketed dressings that were evaluated are presented in Table 5. FIG. 9 shows the average amount of chlorite remaining in these sponges (determined using HPLC) over time after pre-treatment with 0.1 M Na₂CO₃ and dosed with Oxovasin™. The sodium carbonate wash-resistant dressings (i.e. those that remained intact) were the gauze sponge from Johnson&Johnson (rayon/polyester, sponge “C”), the gauze pad from Johnson&Johnson (rayon/polyester/cellulose, sponge “D”) and the latex-free sterile pads from RiteAid (sponge “G”).

The overall results of these further studies were as follows: (i) a pre-treatment with a base such as sodium carbonate, was optimal for obtaining stable products—washing with water caused a rapid drop in pH; (ii) from the polyurethane-based sponges, Medisponge™ 30W and 50P showed the optimal stability at room temperature; (iii) the addition of arginine to the pre-treatment solution reduced color formation; and (iv) application of Oxovasin™ to dried, pre-treated sponges provided optimal stability.

### Example 4

### Evaluation of Pre-Treatment Processes on Medisponge™ 30W

Various pre-treatment processes were evaluated for their effect on the stabilization of Oxovasin™ in Medisponge™ 30W from Lendell Manufacturing. The pre-treatment processes assessed were as follows:
A. 0.1 M Na₂CO₃ solution;
B. Ethanol followed by water;
C. 0.1 M Na₂CO₃ solution followed by ethanol and water; and
D. Ethanol and water followed by 0.1 M Na₂CO₃ solution.
   At the end of the pre-treatment process, the sponges were either dried or excess solution was squeezed. This was followed by addition of a known amount of Oxovasin™ to the sponges. The pH of the sponges was followed in a time dependent manner and the results are provided in Table 6. Note that treatments A and D were optimal.

### Example 5

### Color-Inhibiting Treatments

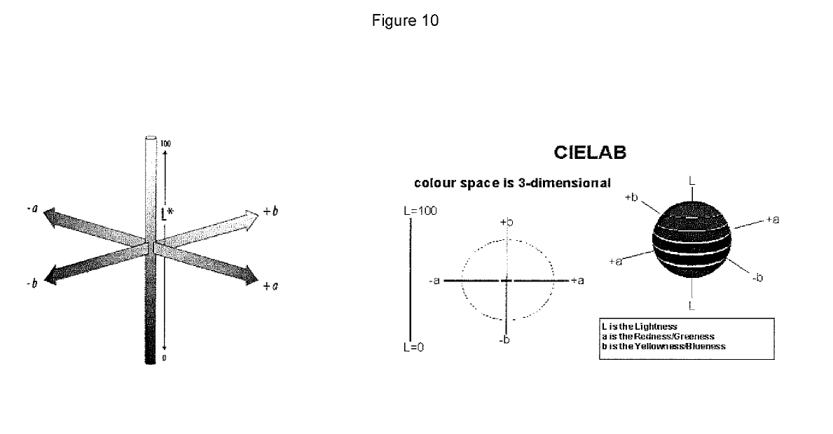

Color measurements were also made on the resulting sponges. The measurements were performed using a Minolta CR300 Chroma meter. The instrument was calibrated using a white calibration plate. The measurements were taken according to the general procedures provided by the manufacturer and the results were provided at CIE L a*b* scale (see FIG. 10). The decrease in color formation of some samples was remarkable as demonstrated by the diminution of L values >100 and increase in b* values <+.

The results for color measurements are summarized in Tables 7A-B for dressings based on substrate 30W (treated with 0.1M sodium carbonate and stored at RT for 6 months (A); treated with sodium carbonate/arginine (1:1 mixture of 0.1 M solution each) at RT for 6 months (B)) and Tables 7C-D for dressings based on substrate 50P (treated with 0.1M sodium carbonate and stored at RT for 12 weeks (C); treated with sodium carbonate/arginine (1:1 mixture of 0.1M solution each) (D) and stored at RT for 16 weeks). The data in Tables 7A-B indicates that the L*, a* and b* values did not change significantly at the early time points (up to 3 months). However at later time points (after 4 months) values at the b* coordinates increased and this is reflected as a slight change of white to light beige hue. The use of sodium carbonate/arginine solution appears to reduce the color shift. Chroma meter measurements for dressings fabricated from Medisponge 50P provided in Tables 7C-D exhibit similar trend to those observed with 30W-based dressings with little change in L*, a* and b* values at early time points. However, a deviation in b* values at later time points reflecting a change in color from white to light beige hue is observed with the sodium carbonate wash and again use of a sodium carbonate/arginine solution lessens the degree of color shift at the foam surface.

### Example 6

### Release Studies

### Preparation of pH 10.8 Carbonate Buffer and Sponge Pre-Treatment

4.2 g sodium carbonate was accurately weighed into a suitable volumetric flask (1000 mL). The material was dissolved in ˜500 mL purified water USP. The pH of the resulting solution was adjusted to ˜10.8 by slowly adding 0.1M sodium hydroxide solution under constant stirring. The flask was filled up to a volume 1 liter with purified water USP and mixed thoroughly resulting in a 0.1 M Na₂CO₃ solution. Mirasorb gauze G, Medisponge™ 50P and Medisponge™ 30W were pretreated with 0.1 M Na₂CO₃ and squeezed. Also tested was Mirasorb gauze G that received no pretreatment with 0.1 M Na₂CO₃. The unpretreated and pretreated Mirasorb gauzes were given experimental names G1 and GW2, respectively. Dosing with Oxovasin™ was performed as discussed below.

### Preparation for Release Studies

Franz diffusion cells (“FDCs”) with a 3 ml receptor volume and 0.55 cm² donor areas were used. Pieces of 30W and 50P foams were punched to fit the opening of donor cell. Each piece of foam was weighed separately and weights were recorded. Nylon membrane filters of approximately 2 cm diameter were punched from stock material (Whatman NL 169, 0.2 μm pore size and NL 17, 0.45 μm pore size, 47 mm diameter, lot #10414012) and used as release membranes. Studies reported here used the 0.2 μm pore size.

### Preparation of Franz Cells

Receptor compartments of FDCs were filled with the sodium carbonate buffer, pH 10.8, and a small stirrer bar was introduced. The Nylon release membranes were mounted on the top of receptor compartments. Receptor and donor compartments were clamped together with uniform pressure using a pinch clamp. Any excess release membrane showing around the edge of the FDC was trimmed with a pair of stainless steel scissors.

Foams 30W and 50P and Mirasorb gauze (GW2 and G1) were placed to the top of the release membrane and the amount of Oxovasin™ solution corresponding to 6 or 8 fold of the weight of 0.1 M sodium carbonate treated sponge was introduced to the top of sponges with a Hamilton type syringe and the foams were slightly tapped to ensure a good contact with the release membrane.

The FDCs were placed in dry blocks equipped with magnetic stirrers and maintained at 25 C with continuous stirring. Measurements were made in 5-fold replicate.

### Sampling

Using a graduated Hamilton type injector syringe, a 300 μL of sample was taken from the sampling port of each Franz cell at 0.5, 1, 2, 3, 4, and 6 hr time points and placed in capped HPLC vials and stored at refrigerated conditions prior to HPLC analysis. At each time point 300 μL of fresh buffer solution was added to the receptor cell.

### Results

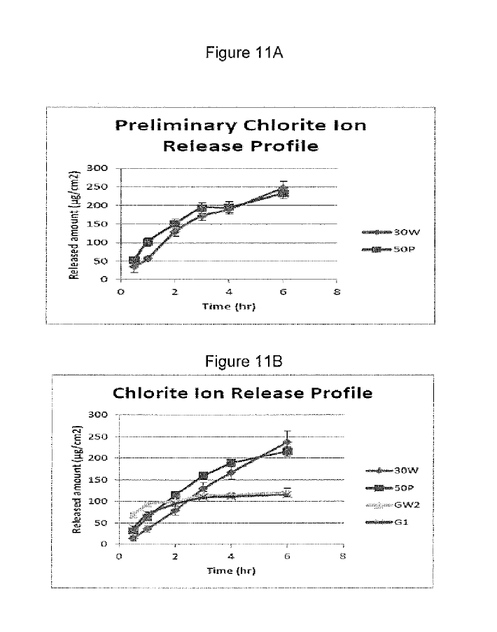

Chlorite concentration was assayed by HPLC. Results for the release of chlorite ion from foam 30W and foam 50P are presented in FIG. 11A. FIG. 11B presents results for 30W, 50P and Mirasorb Gauze GW2 and G1. The Mirasorb gauzes are mechanically robust and able to withstand the pretreatment steps. Greater release of chlorite from the Mirasorb Gauze (GW2 and G1) compared with 30W and 50P foams is observed at early time points, although this trend is reversed at later time points. Table 8 shows the amount of chlorite released from substrates tested in FIG. 11B expressed as an absolute amount (μg/cm²) and as a fraction of the amount of chlorite in the dressing (%).

### Example 7

### Freeze Drying Study

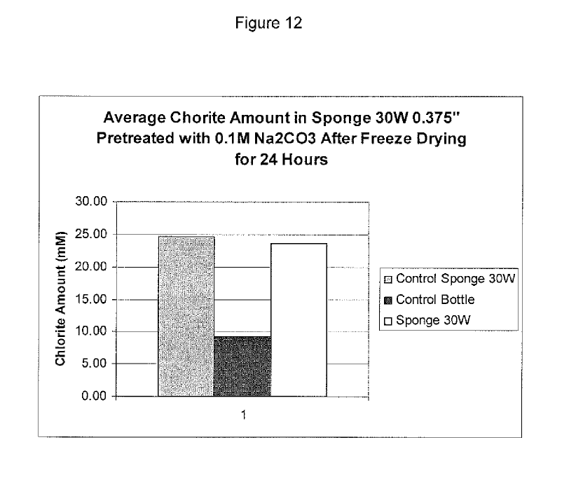

A Medisponge™ 30W 0.375 inches pre-treated with 0.1 M Na₂CO₃ and dosed with a OXO-K993 solution that had been diluted 4× was freeze-dried for 24 hours in a Labconco instrument. The amount of chlorite in this sponge was compared to control sponges similarly treated but kept at room temperature for 24 hours and to chlorite solution in an open container and freeze dried. The results are shown in FIG. 12. The studies showed that loss of chlorite from freeze dried sponges was minimal.

### Example 8

### Evaluation of Pre-Treatment with a Weak Base (0.01M PBS)

The stability of sponges pretreated with a weak base, phosphate buffered saline, was evaluated in this study.

### Method

Medisponge 30W was cut in 2×2 cm square pieces and weighed. The substrates were washed by immersing in 0.01 M PBS at ˜pH 8.5, 9.0 and 9.5 (prepared by diluting PBS 10× concentrate to 1× with deionized water and adjusting the pH using 5M NaOH) for 10 minutes at room temperature. This was followed by squeezing out excess pretreatment solution with a hand roller.

After pretreatment, Oxovasin™ solution (10 times weight of sponge) was evenly added to the sponge surface. The surface pHs of the Oxovasin™ impregnated foams were measured and the foams were placed in pouches made from four layer laminate and sealed. Pouches were stored at RT over a period of up to 10 weeks. The pouches were opened and the surface pH of the foams measured at predetermined intervals.

### Results

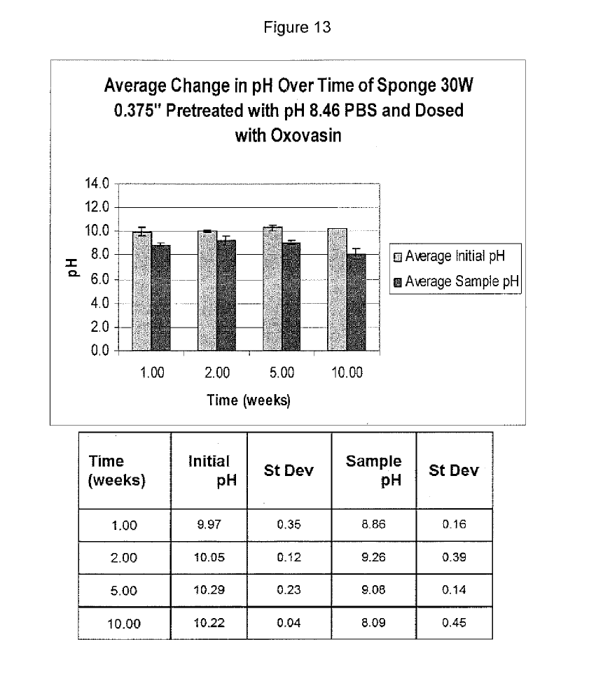

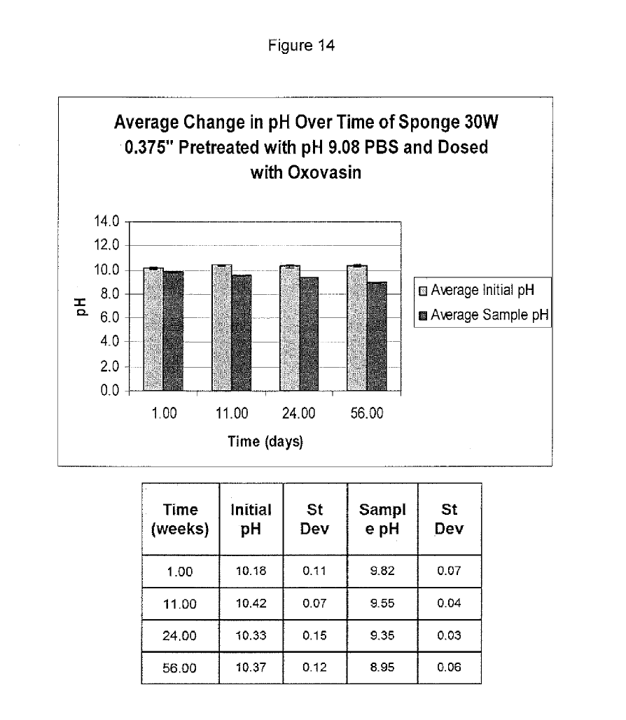

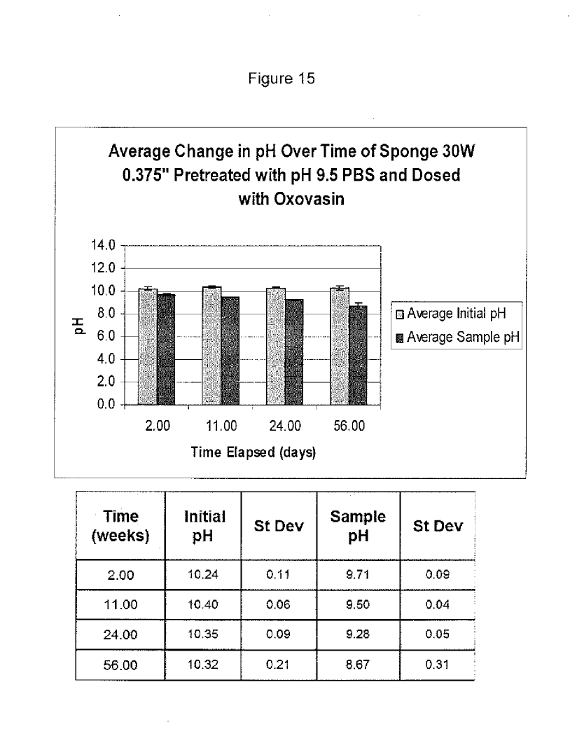

The results are presented in FIGS. 13-15. The drop in surface pH after 1 day, 2 days and 1 week (FIGS. 14, 15 and 13, respectively) of contact time between Oxovasin™ and the PBS treated sponges suggests that chlorite ions in the Oxovasin™ are not stable. Therefore pretreatment of sponges with weak bases is not effective in stabilizing Oxovasin™ at the required pH of greater than or equal to about 10.

### Example 9

### Evaluation of Pre-Treatment with a Weak Base (0.1 M PBS)

The stability of sponges pretreated with a weak base, phosphate buffered saline, was evaluated in this study.

### Materials

Foam compresses (sponges) were Medisponge 30W, size 10×10 cm and about 5 mm thick and were obtained from Filtrona, USA. The laminate foil was A78-polyester paper LDPE/foil LOPE obtained from Firma Beacon Converters, USA. The Oxovasin solution was batch 1189 with a validity until January 2015.

### Preparation of Buffer Solutions

0.1M Phosphate buffered saline (PBS) solution was prepared using sodium dihydrogen phosphate monohydrate (FW 138; 3.45 g=0.025 mol). The details for the preparation of the 0.1 M PBS buffers used are shown in Table 9.

The amount of substance stated in the respective section of Table 9 was dissolved in about 150 g of water. With magnetic stirring, the target pH (±0.05) was adjusted using 1N sodium hydroxide solution; alternatively 0.1N sodium hydroxide solution if pH changes occurred too rapidly. The solution was filled up to 250 g (±0.5 g) using water.

### Sample Preparation

Eight (8) foam compresses (10×10 cm, thickness 5 mm) were cut into pieces of about 2×2 cm (=25 pieces cut from 1 compress) with the help of a paper cutting device. The laminate foil was cut into 200 pieces of about 5×20 cm and the single pieces were folded crossways and welded together on both of the longer sides. The final pouch now had a single opening of about 4.5 cm.

Fifteen (15) foam samples (sponges) were prepared for each group (A, B and C, see Table 9). Five (5) additional samples were prepared using water instead of buffer solution. Each sample was weighed and placed into the respective buffer solution. After 10 minutes, the sample was taken out with forceps and squeezed out on a canted plastic board using a hand roller. The sample was then weighed on a watch glass and Oxovasin was dropped onto the foam until saturation. The excess of Oxovasin™ was discharged. The pH of the surface was then measured and recorded. The sponge was taken with forceps and placed into the foil pouch and the pouch was closed by heat sealing.

The sealed pouches were stored under controlled conditions in a storage room kept at 22° C. The temperature was recorded with an electronic data logger.

### Test Program and Time Intervals

After 1, 2, 5 and 10 weeks of storage, 3 pouches of each group were opened. The sponge was taken out of the pouches with forceps and weighed. The pH of the surface was measured using a pH meter (pMX 30000/ION) and a surface electrode (Mettler-Toledo, LoT403-M8-S7/120).

### Results

### Effect of PBS Buffer 0.1M on the pH Stability of Oxovasin-Treated Sponges

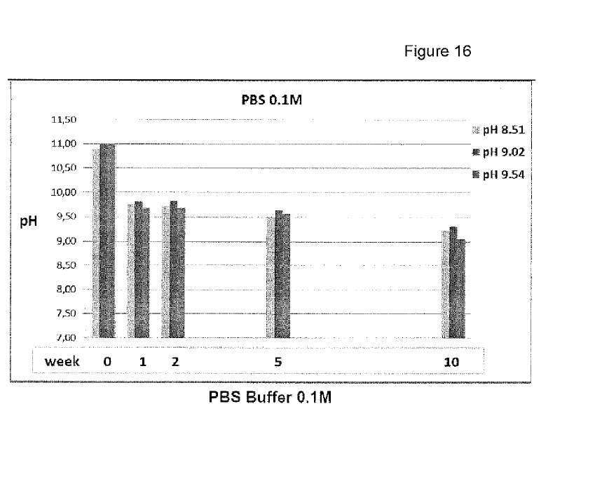

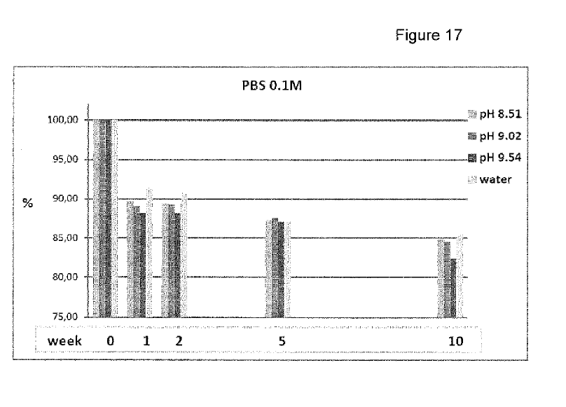

The pH of the sponges dropped by about 10% after 1 week and decreased continually up to 15% after 10 weeks and the drop was independent of the initial pH of the buffer (see FIG. 16 and Table 10). There was no benefit of pretreatment with PBS buffer solution compared to a pretreatment with water (see FIG. 17).

### Example 10

### Evaluation of Enclosure System on Moisture Loss

Sponges were pretreated and dosed with Oxovasin™ as generally outlined in Example 9.

Average Weights Before Storage

The sponges were treated with buffer solution and soaked with Oxovasin™. The average weight of the dry sponges (MV±SD) was 0.223±0.002 g. The average weight of the wet sponges (MV±SD) was 0.696±0.006 g. The average weight of the wet sponges plus Oxovasin™ (MV±SD) was 2.813±0.035 g. Therefore, on average, about 75% of the weight of the final product was made up of Oxovasin™ and 17% of the buffer solution, the latter leading to a pronounced enhancement of the buffer capacity of Oxovasin™.

### Average Weights During Storage

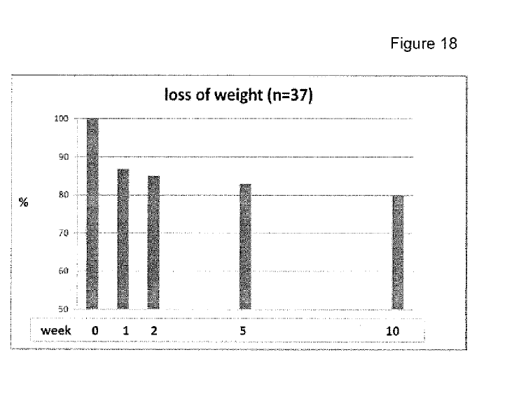

The pouches containing the sponges were opened 1, 2, 5 and 10 weeks after storage (n=37 per time point). Although the non-absorbed Oxovasin™ solution had been removed before the patch was placed until the pouch, a certain amount of liquid was noted after opening the pouches. The average weight of the foam had decreased about 13% after 1 week, and up to 20% after 10 weeks (see FIG. 18).

### Example 11

### Further Evaluation of Pre-Treatment with a Weak Base

Medical grade Medisponge 30W and 50P sponges to be tested were cut in ˜2×2 cm square pieces and weighed (thicknesses of 30W and 50P sponges were 0.250 inches and 0.375 inches, respectfully). The sponges were washed by immersing in PBS for 10 minutes at room temperature. This was followed by squeezing out excess pretreatment solution with a hand roller (wet condition) or squeezing out excess pretreatment solution with a hand roller and drying the sponges at 45° C. in an incubator overnight (dry condition). Typical dry weights of the sponges before treatment are presented in Table 11. Representative weights of the sponges after squeezing excess pretreatment solution from the sponges are provided in Table 12.

After the pretreatment of the sponges (wet conditions and dry conditions), the surface pH was measured and Oxovasin™ solution (10 times the weight of the sponge) was evenly added to the sponge surface. The surface pHs of the Oxovasin impregnated sponges were measured and the sponges were immediately placed in aluminum pouches made from four layer laminate (from exterior to interior, layers of laminate were polyurethane, paper, aluminum with the final contact surface being polyethylene) and sealed. The pouches were stored at RT (temperature conditions were uncontrolled) for 70 hours. The pouches were opened and the surface pH of the sponges measured.

The surface pH of the sponges following PBS treatment was 7.05 for 30W and 7.37 for 50P. After addition of Oxovasin, the surface pH increased to 10.38 for 30W and 10.9 for 50P. Following 70 hours of exposure to Oxovasin, the surface pH of the sponges decreased to 9.56 for 30W and 9.91 for 50P.

The drop in surface pH of the sponges after 70 hours of contact time of the Oxovasin with the PBS-treated sponges suggests that chlorite ion in the Oxovasin is not stable on the PBS-treated sponges. Therefore pretreatment of the sponges with PBS is not effective in stabilizing Oxovasin on the sponges.

### Example 12

### Microbiological Testing

Microbiological testing was carried out with foam/sponge 30W, foam/sponge 30W pretreated with 0.1M sodium carbonate, pretreated foam/sponge 30W impregnated with Oxovasin™.

Details of the foam pieces tested were as follows:
The foam pieces were 1×1 inches;
No special care was taken to avoid micro contamination;
All treatments were performed under the biosafety cabinet (BioGuard, Sanford);
Oxovasin™ amount applied was 10 times foam weight;
The wet fabrication process was used;
Samples were packaged in pouches fabricated from the Conversion Manufacturing laminate.

Microbiological testing was conducted at Ultimate Labs, San Diego, Calif. and carried out according to USP <51> Antimicrobial Effectiveness Test. The results are shown in Tables 13A-13C. Tables 13A-13C show the antimicrobial effectiveness of sponges (no treatment, 0.1 M Na₂CO₃ pre-treatment, and 0.1 M Na₂CO₃ pretreatment followed by dosing with Oxovasin) carried out according to microbiological testing. The first replicate of three is shown.

### Results

The data indicates that the pretreated foam/sponge 30W impregnated with Oxovasin™ did not support microbial proliferation when tested under USP <51>.

The relevant portions of all publications, patents and patent applications are herein incorporated by reference to the same extent as if each individual publication, patent or patent application was specifically and individually indicated to be incorporated by reference. Where a term in the present application is found to be defined differently in a document incorporated herein by reference, the definition provided herein is to serve as the definition for the term.

TABLE 1A

| Substrate | | | |
|---|---|---|---|
| Reference | | | Substrate |
| number | Type | Manufacturer* | Parameters |
| 1 | Medisponge ™ | Lendell | 30W SOO-T |
| | | Manufacturing Inc | (skived) |
| 2 | Medisponge ™ | Lendell | 100 SOO-M |
| | | Manufacturing Inc | |
| 3 | Medisponge ™ | Lendell | 50M PSS-T |
| | | Manufacturing Inc | |
| 4 | Medisponge ™ | Lendell | 30N SOO-O |
| | | Manufacturing Inc | (skived) |
| 5 | Medisponge ™ | Lendell | 01 SOO-O |
| | | Manufacturing Inc | (skived) |
| 6 | Medisponge ™ | Lendell | 50P PSS-M |
| | | Manufacturing Inc | |
| 7 | Medisponge ™ | Filtrona Porous | w/PU film (Thermo |
| | | Technologies | Lamination |
| 8 | Medisponge ™ | Filtrona Porous | 50PW (Poured to |
| | | Technologies | Thickness) |
| 9 | Medisponge ™ | Filtrona Porous | MSM (MDI Based) |
| | | Technologies | (skived to thickness) |
| 10 | Hydrophilic | INOAC | SAQ-X3 iso- |
| | Wound Dressing | Inos Technologies, | 10993 compliant |
| | Foam | LLC | |
| 11 | Medical Grade | Rynel | Rynel 562B 5 mm |
| | Foam | | |
| 12 | TRIAQUA | TRIAQUA | TRIAQUA |
| A | Suprasorb ™ M | Lohmann Rauscher | PU membrane |
| B | Suprasorb ™ P | Lohmann Rauscher | PU foam dressing |
| | | | self adhesive |
| C | Suprasorb ™ H | Lohmann Rauscher | Hydrocolloid |
| | | | would dressing std |
| D | Unknown | Unknown | Foamex 14321872 |
| E | Unknown | Unknown | Foamex 14321539 |
| F | Medical Grade | RYNEL | Rynel 562-6 5 mm |
| | Foam | | |
| G | Unknown | Unknown | Cotton medical |
| | | | fabric |
| H | Unknown | Unknown | Nonwoven medical |
| | | | fabric |
| J | Unknown | Unknown | Mesh fabric (3 |
| | | | layers) |

*Note that Lendell was acquired by Filtrona while these studies were conducted

TABLE 1B

| Ingredient | Supplier |
|---|---|
| Sodium carbonate NF | Spectrum |
| Potassium carbonate USP | Spectrum |
| Arginine USP | Calbiochem |
| Water USP | Thermo Scientific |
| Hydroxypropyl cellulose HY117 NF | Spectrum |
| Madisponge 30W | Filtrona |
| Medisponge 50P | Filtrona |
| Stratex 7.9NLYB-E | DelStar Technologies, Inc. |
| Mirasorb gauze | Johnson & Johnson |
| Baby wipes | Equate |
| 4 layer laminate, A78 | Conversion Manufacturers Inc |

TABLE 2

| Sponge # | Treatment | Condition | % remaining |
|---|---|---|---|
| 6 | Na2CO3 | Dry | 87.7 |
| 6 | Na2CO3 | Wet | 52.13 |
| 6 | Na2CO3/Arginine | Dry | 95.82 |
| 6 | Na2CO3/Arginine | Wet | 58.55 |
| 1 | Na2CO3 | Dry | 95.43 |
| 1 | Na2CO3 | Wet | 53.13 |
| 1 | Na2CO3/Arginine | Dry | 99.77 |
| 1 | Na2CO3/Arginine | Wet | 64.73 |

TABLE 3

| | | Time point | pH after | | Measured | |
|---|---|---|---|---|---|---|
| Treatment | Condition | (week) | dosing | StDEev | pH | StDev |
| 0.1M Na2CO3 | RT | 2 | 10.92 | 0.06 | 10.47 | 0.36 |
| 0.1M Na2CO3 | RT | 4 | 10.95 | 0.04 | 10.66 | 0.11 |
| 0.1M Na2CO3/Arginine | RT | 2 | 10.92 | 0.02 | 10.25 | 0.04 |
| 0.1M Na2CO3/Arginine | RT | 4 | 10.93 | 0.01 | 10.01 | 0.14 |
| 0.1M Na2CO3 | Refrigerator | 2 | 10.91 | 0.05 | 10.65 | 0.09 |
| 0.1M Na2CO3 | Refrigerator | 4 | 10.89 | 0.04 | 10.73 | 0.05 |
| 0 1M Na2CO3/Arginine | Refrigerator | 2 | 10.90 | 0.03 | 10.13 | 0.23 |
| 0.1M Na2CO3/Arginine | Refrigerator | 4 | 10.89 | 0.03 | 10.18 | 0.13 |

TABLE 4

| | | Chlorite | | Chlorite | |
|---|---|---|---|---|---|
| | | amount | | amount | |
| | | (mM)/2 | | (mM)/4 | |
| Condition | Wash | weeks | StDev | weeks | StDev |
| Room | 0.1M | 12.60 | 0.21 | 12.56 | 0.63 |
| Temper- | Na2CO3 | | | | |
| ature | | | | | |
| Room | 0.1M | 12.78 | 0.21 | 12.75 | 0.40 |
| Temper- | Na2CO3/ARG | | | | |
| ature | | | | | |
| Refrig- | 0.1M | 12.55 | 0.58 | 12.16 | 0.33 |
| erated | Na2CO3 | | | | |
| Refrig- | 0.1M | 12.84 | 0.13 | 12.01 | 0.29 |
| erated | Na2CO3/ARG | | | | |

TABLE 5

| Sponge | | Manufac- | | Composi- |
|---|---|---|---|---|
| Reference | Type | turer | Parameters | tion |
| A | Surgical | Rite Aid | Sterile | Latex |
| | Dressing | | 5″ × 9″ Pad | Free |
| B | Surgical | Johnson & | Sterile | N/A |
| | Dressing, | Johnson | 5″ × 9″ Pad | |
| | “Surgipad” | | | |
| C | Gauze Sponge, | Johnson & | Sterile | Rayon/ |
| | “Mirasorb” | Johnson | 4ply, 4″ × 4″ | Polyester |
| | | | | Blend |
| D | Gauze Pad | Johnson & | Sterile | Rayon/ |
| | | Johnson | 3″/po × 3″/po | Polyester |
| | | | | Cellulose |
| | | | | Blend |
| E | Dressing | Rite Aid | Sterile | Latex |
| | Sponges | | 4″ × 4″ Dressing | Free |
| F | Triple Layer | Johnson & | Sterile | N/A |
| | Non-stick Pad | Johnson | 2″/po × 3″/po | |
| G | Sterile Pads | Rite Aid | Sterile | Latex |
| | | | 4″ × 4″ | Free |

TABLE 6

| | | | | Mass | Mass of | | | | |
|---|---|---|---|---|---|---|---|---|---|
| | | | | after | Oxovasin | | | Time | pH at |
| | | | Mass | Dosing | Used | pH after | pH after | Elapsed | Time |
| | Wash | Replicate | (mg) | (mg) | (mg) | Wash | Dosing | (Weeks) | Point |
| Dry | A | 1 | 259.11 | 2442.42 | 2183.31 | 11.05 | 10.89 | 1 | 10.86 |
| | | 2 | 260.03 | 2498.48 | 2238.45 | 11.11 | 11.01 | 2 | 10.84 |
| | | 3 | 260.80 | 2590.67 | 2329.87 | 11.21 | 11.05 | 3 | 10.82 |
| | B | 1 | 213.69 | 2142.22 | 1928.53 | 7.32 | 10.99 | 1 | 9.79 |
| | | 2 | 232.54 | 2296.38 | 2063.84 | 7.53 | 10.99 | 2 | 9.43 |
| | | 3 | 235.79 | 2332.14 | 2096.35 | 8.23 | 10.90 | 3 | 9.44 |
| | C | 1 | 211.19 | 2167.38 | 1956.19 | 8.58 | 10.77 | 1 | 9.9 |
| | | 2 | 263.80 | 2394.44 | 2130.64 | 7.46 | 10.93 | 2 | 9.46 |
| | | 3 | 260.60 | 2473.74 | 2213.14 | 7.60 | 10.83 | 3 | 9.84 |
| | D | 1 | 351.86 | 2902.55 | 2550.69 | 9.58 | 10.37 | 1 | 10.54 |
| | | 2 | 231.92 | 2242.36 | 2010.44 | 10.07 | 10.79 | 2 | 10.54 |
| | | 3 | 222.59 | 2245.10 | 2022.51 | 10.40 | 10.92 | 3 | 10.39 |
| Wet | A | 1 | 2351.86 | 3838.48 | 1486.62 | 11.20 | 10.88 | 1 | 10.88 |
| | | 2 | 2040.63 | 3394.31 | 1353.68 | 11.19 | 10.91 | 2 | 10.73 |
| | | 3 | 2022.09 | 3214.65 | 1192.56 | 11.22 | 10.76 | 3 | 10.76 |
| | B | 1 | 1801.35 | 3275.55 | 1474.20 | 7.83 | 10.60 | 1 | 9.17 |
| | | 2 | 2329.47 | 3619.07 | 1289.60 | 8.32 | 10.83 | 2 | 8.82 |
| | | 3 | 1628.83 | 3160.77 | 1531.94 | 7.10 | 10.86 | 3 | 9.01 |
| | C | 1 | 2162.96 | 3136.16 | 973.20 | 8.02 | 10.99 | 1 | 9.65 |
| | | 2 | 2281.60 | 3408.86 | 1127.26 | 7.32 | 10.92 | 2 | 9.23 |
| | | 3 | 2237.93 | 3490.50 | 1252.57 | 7.15 | 10.93 | 3 | 9.27 |
| | D | 1 | 1751.26 | 2943.86 | 1192.60 | 10.72 | 10.81 | 1 | 10.02 |
| | | 2 | 2051.82 | 3276.74 | 1224.92 | 10.67 | 10.45 | 2 | 9.75 |
| | | 3 | 2610.13 | 3464.39 | 854.26 | 10.84 | 10.79 | 3 | 10 |

TABLE 7A

| Time | L*a*b after dosing | | | L*a*b at time point | | |
|---|---|---|---|---|---|---|
| (weeks) | L* | a* | b* | L* | a* | b* |
| 1 | 91.60 | −1.48 | 3.14 | 91.92 | −1.23 | 3.17 |
| 2 | 91.95 | −1.35 | 2.75 | 91.29 | −1.33 | 3.06 |
| 3 | 92.52 | −1.15 | 2.14 | 91.63 | −1.40 | 3.39 |
| 4 | 92.36 | −1.21 | 2.19 | 92.40 | −0.82 | 3.38 |
| 8 | 91.47 | −1.29 | 2.50 | 89.38 | −1.41 | 4.72 |
| 12 | 91.35 | −1.45 | 3.40 | 91.73 | −1.45 | 6.84 |
| 16 | 91.42 | −1.10 | 2.03 | 91.41 | −1.15 | 6.74 |
| 20 | 91.49 | −1.23 | 2.60 | 89.12 | −1.10 | 7.37 |
| 24 | 92.06 | −1.08 | 2.16 | 92.66 | −0.65 | 9.44 |

TABLE 7B

| Time | L*a*b after dosing | | | L*a*b at time point | | |
|---|---|---|---|---|---|---|
| (weeks) | L* | a* | b* | L* | a* | b* |
| 1 | 92.22 | −1.25 | 1.26 | 91.70 | −1.18 | 1.40 |
| 2 | 92.66 | −1.42 | 1.71 | 90.75 | −1.43 | 1.77 |
| 3 | 92.90 | −1.26 | 1.26 | 90.71 | −1.31 | 1.24 |
| 4 | 92.49 | −1.27 | 1.50 | 91.20 | −1.37 | 1.43 |
| 8 | 92.36 | −1.20 | 0.90 | 91.02 | −1.27 | 1.20 |
| 12 | 92.13 | −1.27 | 1.18 | 91.88 | −1.35 | 1.83 |
| 16 | 92.41 | −1.27 | 1.14 | 93.53 | −1.33 | 2.20 |
| 20 | 92.56 | −1.15 | 1.00 | 93.27 | −1.22 | 2.19 |
| 24 | 92.29 | −1.25 | 1.12 | 92.77 | −1.32 | 2.39 |

TABLE 7C

| Time | L*a*b after dosing | | | L*a*b at time point | | |
|---|---|---|---|---|---|---|
| (weeks) | L* | a* | b* | L* | a* | b* |
| 1 | 89.19 | −0.80 | 0.74 | 91.21 | −0.77 | 1.78 |
| 2 | 88.97 | −0.86 | 1.36 | 92.23 | −1.11 | 2.69 |
| 3 | 85.76 | −0.72 | 1.13 | 92.04 | −0.98 | 2.92 |
| 4 | 86.04 | −0.67 | 0.89 | 92.50 | −0.94 | 3.14 |
| 8 | 85.70 | −0.61 | 0.95 | 91.30 | −0.84 | 4.70 |
| 12 | 87.60 | −0.63 | 1.03 | 91.52 | −0.96 | 5.34 |

TABLE 7D

| Time | L*a*b after dosing | | | L*a*b at time point | | |
|---|---|---|---|---|---|---|
| (weeks) | L* | a* | b* | L* | a* | b* |
| 1 | 86.02 | −1.14 | 1.49 | 90.82 | −1.18 | 1.18 |
| 2 | 83.87 | −1.16 | 1.06 | 90.82 | −1.21 | 0.93 |
| 3 | 82.68 | −0.88 | 1.08 | 91.25 | −1.19 | 1.20 |
| 4 | 83.28 | −1.30 | 1.35 | 92.08 | −1.09 | 1.24 |
| 8 | 85.39 | −0.89 | 0.83 | 91.98 | −1.03 | 1.08 |
| 12 | 82.80 | −1.09 | 1.71 | 92.48 | −1.39 | 2.28 |
| 16 | 83.42 | −1.20 | 1.72 | 91.34 | −1.25 | 1.84 |

TABLE 8

| | 50W | | 50P | | GW2 | | G1 | |
|---|---|---|---|---|---|---|---|---|
| Time | μg/cm² | % | μg/cm² | % | μg/cm² | % | μg/cm² | % |
| 0.5 hrs | 13.52 | 0.3 | 30.70 | 1.3 | 68.54 | 7.6 | 38.81 | 4.2 |
| 1 hrs | 35.50 | 0.8 | 62.22 | 2.6 | 95.11 | 10.6 | 71.71 | 7.8 |
| 2 hrs | 79.00 | 1.7 | 115.30 | 4.7 | 100.26 | 11.1 | 95.01 | 10.3 |
| 3 hrs | 130.11 | 2.8 | 159.98 | 6.6 | 115.89 | 12.9 | 109.87 | 11.9 |
| 4 hrs | 167.20 | 3.6 | 188.94 | 7.8 | 114.46 | 12.7 | 110.96 | 12.0 |
| 6 hrs | 237.20 | 5.1 | 215.58 | 8.9 | 122.19 | 13.6 | 115.92 | 12.5 |

TABLE 9
PBS Buffer 0.1M

| Group | Weight (g) | Target pH | Measured pH | Final weight (g) |
|---|---|---|---|---|
| A | 3.4505 | 8.5 | 8.51 | 250.03 |
| B | 3.4517 | 9.0 | 9.02 | 250.02 |
| C | 3.4512 | 9.5 | 9.54 | 250.03 |

TABLE 10

| pH of buffer | pH at start | After 1 week | After 2 weeks | After 5 weeks | After 10 weeks |
|---|---|---|---|---|---|
| 8.51 | 10.88 ± 0.03 | 9.76 ± 0.13 | 9.72 ± 0.03 | 9.50 ± 0.11 | 9.22 ± 0.16 |
| 9.02 | 11.01 ± 0.04 | 9.80 ± 0.02 | 9.83 ± 0.02 | 9.64 ± 0.05 | 9.30 ± 0.12 |
| 9.54 | 10.99 ± 0.04 | 9.69 ± 0.10 | 9.69 ± 0.03 | 9.57 ± 0.07 | 9.06 ± 0.18 |
| n/group | 12 | 3 | 3 | 3 | 3 |

TABLE 11

| | Weight 50P | Weight 30W |
|---|---|---|
| Substrate | 0.250 in (g) | 0.375 in (g) |
| #1 | 0.2597 | 0.4757 |
| #2 | 0.2943 | 0.4784 |
| #3 | 0.2819 | 0.4964 |
| #4 | 0.2467 | 0.4228 |
| #5 | 0.2938 | 0.5234 |
| Average | 0.27528 | 0.47934 |
| Standard | 0.0190 | 0.0330 |
| Deviation | | |

TABLE 12

| | 50P (0.25 | 50P (0.25 | 30W (0.375 | 30W (0.375 |
|---|---|---|---|---|
| | in) post | in) fluid | in) post | in) fluid |
| Substrate | squeeze (g) | weight (g) | squeeze (g) | weight (g) |
| #1 | 1.0829 | 0.8232 | 1.5857 | 1.11 |
| #2 | 1.2692 | 0.9749 | 1.6554 | 1.177 |
| #3 | 1.0683 | 0.7864 | 1.6775 | 1.1811 |
| Average | 1.1401 | 0.8615 | 1.6395 | 1.1560 |
| Standard | 0.1120 | 0.0999 | 0.0479 | 0.0399 |
| Deviation | | | | |

TABLE 13A
Product - Sponge/No Treatment - Replicate 1 - Dilution 1:100
Plate Count Method

| | Initial | | | | | | |
|---|---|---|---|---|---|---|---|
| | Inoculum | Day 7 | Log | Day 14 | Log | Day 28 | Log |
| Organism | (CFU/mL) | (CFU/mL) | Reduction | (CFU/mL) | Reduction | (CFU/mL) | Reduction |
| A. Niger | 74,640,000 | 82,400,000 | 0 | 95,200,000 | 0 | 103,100,000 | 0 |
| C. Albicans | 81,260,000 | 92,200,000 | 0 | 103,100,000 | 0 | 111,300,000 | 0 |
| E. Coli | 108,080,000 | 117,200,000 | 0 | 124,600,000 | 0 | 132,200,000 | 0 |
| P. Aeruginosa | 118,200,000 | 124,800,000 | 0 | 130,200,000 | 0 | 138,000,000 | 0 |
| S. Aureus | 94,300,000 | 99,200,000 | 0 | 112,900,000 | 0 | 121,400,000 | 0 |

TABLE 13B
Product - MediSponge (0.1M NaCO₃) - Replicate 1 - Dilution 1:100
Plate Count Method

| | Initial Inoculum | Day 7 | Log | Day 14 | Log | Day 28 | Log |
|---|---|---|---|---|---|---|---|
| Organism | (CFU/mL) | (CFU/mL) | Reduction | (CFU/mL) | Reduction | (CFU/mL) | Reduction |
| A. Niger | 74,640,000 | 47,500,000 | 0 | 45,600,000 | 0 | 42,600,000 | 0 |
| C. Albicans | 81,280,000 | 68,800,000 | 0 | 65,300,000 | 0 | 60,200,000 | 0 |
| E. Coli | 108,080,000 | 89,400,000 | 0 | 72,200,000 | 0 | 73,400,000 | 0 |
| P. Aeruginosa | 118,200,000 | 99,300,000 | 0 | 88,700,000 | 0 | 83,500,000 | 0 |
| S. Aureus | 94,300,000 | 76,200,000 | 0 | 72,500,000 | 0 | 68,900,000 | 0 |

TABLE 13C
Product - Pretreated Sponge with Oxovasin ® Replicate 1 - Dilution 1:100
Plate Count Method

| | Initial Inoculum | Day 7 | Log | Day 14 | Log | Day 28 | Log |
|---|---|---|---|---|---|---|---|
| Organism | (CFU/mL) | (CFU/mL) | Reduction | (CFU/mL) | Reduction | (CFU/mL) | Reduction |
| A. Niger | 74,640,000 | 998,000 | 2 | 78,600 | 3 | 45,200 | No Increase |
| C. Albicans | 81,280,000 | 829,000 | 2 | 66,200 | 3 | 23,900 | No Increase |
| E. Coli | 108,080,000 | 756,000 | 3 | 89,200 | 4 | 36,900 | No Increase |
| P. Aereuginosa | 118,200,000 | 888,000 | 3 | 94,500 | 4 | 64,500 | No Increase |
| S. Aureus | 94,300,000 | 912,000 | 2 | 85,400 | 3 | 77,300 | No Increase |

1-69. (canceled)

70. A wound dressing comprising an absorbent material, an effective amount of chlorite and an amount of a base to provide a pH of the absorbent material greater than or equal to about 10.

71. The wound dressing of claim 70, wherein the chlorite added to the absorbent material is a stabilized chlorite composition.

72. The wound dressing of claim 71, wherein the stabilized chlorite composition is OXO-K993, or a composition comprising about 0.01-0.1%, 0.1-1%, 1-10%, 10-20%, 20-30%, 30-50% or 50-90% (w/v) of OXO-K993.

73. The wound dressing of claim 71, wherein the stabilized chlorite composition is a composition comprising Oxovasin™ or a composition comprising about 2% (w/v) OXO-K993.

74. The wound dressing of claim 71, wherein the stabilized chlorite composition is a composition comprising about 2% (w/v) OXO-K993, about 2% (w/v) glycerol and about 96% (w/v) water.

75. The wound dressing of claim 70, wherein the chlorite is a chlorite salt selected from sodium chlorite, potassium chlorite, magnesium chlorite and barium chlorite.

76. The wound dressing of claim 75, wherein the chlorite is added to the absorbent material as a chlorite solution comprising, consisting of or consisting essentially of sodium chlorite.

77. The wound dressing of claim 70, wherein the chlorite is added to the absorbent material as a stabilized chlorite solution comprising, consisting of or consisting essentially of chlorite, chlorate, chloride, sulfate and sodium ions.

78. The wound dressing of claim 70, wherein the base is an inorganic base selected from alkali metal bases, alkaline earth metal bases, ammonia, ammonia hydroxides and mixtures thereof, or an organic base selected from alkylamines, alkylamides, methyloxides, citrates and mixtures thereof, or a mixture of an inorganic and an organic base.

79. The wound dressing of claim 78, wherein the inorganic base is alkali metal base selected from sodium carbonate, pentasodium triphosphate, potassium, pyrophosphate, sodium pyrophosphate, potassium carbonate and mixtures thereof.

80. The wound dressing of claim 79, wherein the alkali metal base comprises, consists of or consists essentially of sodium carbonate or potassium carbonate.

81. The wound dressing of claim 70, wherein the base is added to the absorbent material as a solution with a concentration of about 0.01, 0.05, 0.10, 0.15, 0.20, 0.25, 0.30, 0.35, 0.40, 0.45 or 0.50 M (mol/L), or fractions in between.

82. The wound dressing of claim 70, wherein the absorbent material is treated before, or concurrently with the chlorite, with an excess amount of a base solution and the excess of the base solution and chlorite are removed by squeezing and/or drying.

83. The wound dressing of claim 70, further comprising a color stabilizing agent.

84. The wound dressing of claim 83, wherein the color stabilizing agent is arginine.

85. A method for preparing a wound dressing comprising:
(a) treating an absorbent material with a base solution to provide a pH that is greater than or equal to about 10; and
(b) treating the absorbent material with an effective amount of chlorite,
wherein the absorbent material is treated with the chlorite concurrently with or after treatment with the base.

86. The method of claim 85, wherein the absorbent material is treated with a base to provide a pH of the absorbent material that is about 10, 10.1, 10.2, 10.3, 10.4, 10.5, 10.6, 10.7, 10.8, 10.9, 11.0, 11.1, 11.2, 11.3, 11.4, 11.5, 11.6, 11.7, 11.8, 11.9, 12, 12.1, 12.2, 12.3, 12.4, 12.5, 12.6, 12.7, 12.8, 12.9 or 13.

87. The method of claim 85, wherein the absorbent material is treated with an excess amount of 0.1 M sodium carbonate at room temperature followed by squeezing excess base solution from the material and/or drying.

88. A wound dressing prepared according to the method of claim 85.

89. A method for treating wounds or for treating a condition or an injury comprising applying the wound dressing of claim 70 to a subject in need thereof, wherein the condition or injury is selected from skin diseases, skin disorders (including topical and neuro dermatitis, psoriasis, herpes simplex, herpes zoster and acne), infections, burns and wound healing (including pressure, burn, post-operative and post-traumatic wound healing, as well as chronic wound healing in the case of diabetic ulcers, venous ulcers, arterial ulcers, decubitus ulcers and the like).
